SEQUENCE LISTING (35 sequences)

SEQ ID NO: 1 - DNA, 1614 bp, Actinobacillus succinogenes
  CDS (1)..(1614)
atgactgacttaaacaaactcgttaaagaacttaatgacttagggcttaccgatgttaaggaaattgtgtataacccgagttatgaacaacttttcgaggaagaaaccaaaccgggtttggagggtttcgataaagggacgttaaccacgcttggcgcggttgccgtcgatacggggatttttaccggtcgttcaccgaaagataaatatatcgtttgcgatgaaactacgaaagacaccgtttggtggaacagcgaagcggcgaaaaacgataacaaaccgatgacgcaagaaacttggaaaagtttgagagaattagtggcgaaacaactttccggtaaacgtttattcgtggtagaaggttactgcggcgccagtgaaaaacaccgtatcggtgtgcgtatggttactgaagtggcatggcaggcgcattttgtgaaaaacatgtttatccgaccgaccgatgaagagttgaaaaatttcaaagcggattttaccgtgttaaacggtgctaaatgtactaatccgaactggaaagaacaaggtttgaacagtgaaaactttgtcgctttcaatattaccgaaggtattcagcttatcggcggtacttggtacggcggtgaaatgaaaaaaggtatgttctcaatgatgaactacttcctgccgttaaaaggtgtggcttccatgcactgttccgccaacgtaggtaaagacggtgacgtggctattttcttcggtttatccggtacgggtaaaacaacgctttcgaccgatcctaaacgccaattaatcggtgatgacgaacacggttgggatgaatccggcgtatttaactttgaaggcggttgttacgcgaaaaccattaacttatctcaagaaaacgaaccggatatttacggcgcaatccgtcgtgacgcattattagaaaacgtcgtggttcgtgcagacggttccgttgactttgacgacggttcaaaaacagaaaatacccgtgtttcatatccgatttaccacatcgacaacatcgttcgtccggtatcgaaagccggtcatgcaaccaaagtgattttcttaaccgcggacgcattcggcgtattgccgccggtttcaaaactgactccggaacaaaccgaatactacttcttatccggctttactgcaaaattagcgggtacggaacgcggcgtaaccgaaccgactccgacattctcggcctgtttcggtgcggcattcttaagcctgcatccgattcaatatgcggacgtgttggtcgaacgcatgaaagcctccggtgcggaagcttatttggtgaacaccggttggaacggcacgggtaaacgtatttcaatcaaagatacccgcggtattatcgatgcgattttggacggttcaatcgaaaaagcggaaatgggcgaattgccaatctttaatttagcgattcctaaagcattaccgggtgttgatcctgctattttggatccgcgcgatacttacgcagacaaagcgcaatggcaagttaaagcggaagatttggcaaaccgtttcgtgaaaaactttgtgaaatatacggcgaatccggaagcggctaaattagttggcgccggtccaaaagca

SEQ ID NO: 2 - protein, 538 aa, Actinobacillus succinogenes
MTDLNKLVKELNDLGLTDVKEIVYNPSYEQLFEEETKPGLEGFDKGTLTTLGAVAVDTGIFTGRSPKDKYIVCDETTKDTVWWNSEAAKNDNKPMTQETWKSLRELVAKQLSGKRLFVVEGYCGASEKHRIGVRMVTEVAWQAHFVKNMFIRPTDEELKNFKADFTVLNGAKCTNPNWKEQGLNSENFVAFNITEGIQLIGGTWYGGEMKKGMFSMMNYFLPLKGVASMHCSANVGKDGDVAIFFGLSGTGKTTLSTDPKRQLIGDDEHGWDESGVFNFEGGCYAKTINLSQENEPDIYGAIRRDALLENVVVRADGSVDFDDGSKTENTRVSYPIYHIDNIVRPVSKAGHATKVIFLTADAFGVLPPVSKLTPEQTEYYFLSGFTAKLAGTERGVTEPTPTFSACFGAAFLSLHPIQYADVLVERMKASGAEAYLVNTGWNGTGKRISIKDTRGIIDAILDGSIEKAEMGELPIFNLAIPKALPGVDPAILDPRDTYADKAQWQVKAEDLANRFVKNFVKYTANPEAAKLVGAGPKA

SEQ ID NO: 3 - DNA, 1281 bp, Actinobacillus succinogenes
  CDS (1)..(1281)
atgtcagatttaagtcaaaaagcactcgattttcacgaatttcccgtcccgggtaaaatttccgttacaccgactaaaccattagaaagccaaggcgatttggctcttgcctattctcccggcgttgccgaaccttgcttagccattcaacaggatcctgcaaaatcttatcgttacaccgctcgcggcaatctagtcggcgtaatttccaacggctcggccgtcttgggattaggcaatatcggcgcattggcgggaaaaccggttatggaaggcaaaggcgtattattcaaaaaatttgccggcgttgatgtattcgatatcgaaatcgatgaaagtaatccggaaaaattcgttgaaatcgtcgcggcattagaaccgaccttcggcggcatcaatctcgaagacattaaagcaccggaatgtttctatatcgaaaaagccttacgcgaacgcatgggaattcctgttttccacgatgaccagcacggtaccgccatcatcagttccgcggccgtattaaacggtttacgcattcaaaacaaaaaaatcgaagatgtcaaattagtcgcgtccggtgccggtgcagcctctatcgcatgcctgaatttattaatttcattaggcgtaaaacgcgaaaacattaccgtatgcgactcaaaaggcgtaatctttgaaggccgtgataacaaaatggacgaaaccaaaaaagcctttgcgcaaaaagacaccggagcgcgcacattagcggatgctatttcaaacgccgatgtattcttaggttgttccgccgccggtgcattaacgcctgaaatggtaaaaaccatggcatcccatccgctgattttggcattggctaaccctgatccggaaattactccgcctgaggcgcatgccgctcgtgaagacgctatcgtctgtaccggtcgttcggactatccgaaccaagtaaacaatgtgctttgtttcccgttcattttccgcggcgcattagatgtaggcgcgaccgaaattaacgaagaaatgaaaatggcgaccgttcgcgctatcgccgatttagcattagaagaaccgactgcagaagtgttggcggcttacggcaacaaagccatgtctttcggtccggaatatattattccgactgcatttgattcccgtttaatcacccgtattgcgccggcagtggccaaagcggcaatggacagcggtgtggcaacccgtccgattacggattgggatgactatgcggcgcaattgaaagcgcatagccgtcgtttaaaa

SEQ ID NO: 4 - protein, 427 aa, Actinobacillus succinogenes
MSDLSQKALDFHEFPVPGKISVTPTKPLESQGDLALAYSPGVAEPCLAIQQDPAKSYRYTARGNLVGVISNGSAVLGLGNIGALAGKPVMEGKGVLFKKFAGVDVFDIEIDESNPEKFVEIVAALEPTFGGINLEDIKAPECFYIEKALRERMGIPVFHDDQHGTAIISSAAVLNGLRIQNKKIEDVKLVASGAGAASIACLNLLISLGVKRENITVCDSKGVIFEGRDNKMDETKKAFAQKDTGARTLADAISNADVFLGCSAAGALTPEMVKTMASHPLILALANPDPEITPPEAHAAREDAIVCTGRSDYPNQVNNVLCFPFIFRGALDVGATEINEEMKMATVRAIADLALEEPTAEVLAAYGNKAMSFGPEYIIPTAFDSRLITRIAPAVAKAAMDSGVATRPITDWDDYAAQLKAHSRRLK

SEQ ID NO: 5 - DNA, 936 bp, Actinobacillus succinogenes
  CDS (1)..(936)
atgaaagtaaccttattaggcgccagcggcggtatcggtcaacctctttcattgttgttaaaattacatcttccggcagaaagcgatttaagcttatacgatgttgcgccggtcacccccggtgtggcgaaagacatcagccatattccgacttcggttgaagtggaaggtttcggcggcgatgatccgtccgaggcattaaaaggggcggatatcgttttaatctgtgcgggtgtggcgcgtaagccgggtatgactcgtgcggatttgtttaatgttaacgccggtattatccagaatttagtggaaaaagttgcgcaagtttgcccgcaggcttgtgtttgcattatcactaatccggtgaactcgattattccgattgcggcggaagtgctgaaaaaagcgggcgtatacgataaacggaaattattcggtattactacgctggataccatccgttccgaaaaatttatcgtgcaagcgaaaaatattgaaatcaaccgtaacgatatttcagttatcggcggacattcaggtgtgacgattttacctttgttgtcacaaattccgcatgtggaatttaccgagcaggaattaaaagatttaactcaccgcatccaaaatgccggcaccgaagtggtagaagctaaagccggtgcgggttccgctacactttccatggcgtatgcggcaatgcgttttgtggtttccatggctcgcgcattaaacggcgaagtgattacggaatgcgcctatattgaaggcgacggtaaattcgcccgtttctttgcacaaccggttcgtttgggtaaaaacggcgtagaagaaattctgccgttaggtacattaagcgcatttgagcaacaagcgcttgaagcgatgttaccgaccttgcaaactgacattgataacggtgtgaaatttgttaccggcgaa

SEQ ID NO: 6 - protein, 312 aa, Actinobacillus succinogenes
MKVTLLGASGGIGQPLSLLLKLHLPAESDLSLYDVAPVTPGVAKDISHIPTSVEVEGFGGDDPSEALKGADIVLICAGVARKPGMTRADLFNVNAGIIQNLVEKVAQVCPQACVCIITNPVNSIIPIAAEVLKKAGVYDKRKLFGITTLDTIRSEKFIVQAKNIEINRNDISVIGGHSGVTILPLLSQIPHVEFTEQELKDLTHRIQNAGTEVVEAKAGAGSATLSMAYAAMRFVVSMARALNGEVITECAYIEGDGKFARFFAQPVRLGKNGVEEILPLGTLSAFEQQALEAMLPTLQTDIDNGVKFVTGE

SEQ ID NO: 7 - DNA, 1392 bp, Actinobacillus succinogenes
  CDS (1)..(1392)
atgacatttcgtattgaaaaagacactatgggcgaggttcaagttcctgcggataaatattgggccgcacaaacggaacgttcacgcaataactttaaaatcggacctgcagcgtcaatgccacacgaaattattgaagcattcggttatttgaaaaaagcggccgcttatgccaacgcggatttaggcgtattgccggctgaaaaacgcgatttgatcgcacaagcctgcgatgaaattctggctcgtaaattagatgatcaattcccgttagttatctggcaaaccggttcgggtacacaatccaatatgaatctgaacgaagttatcgctaaccgcgcacatgtgattaacggtggtaaattaggtgaaaaatctattattcatccaaacgacgatgtaaacaaatctcaatcttcaaacgatacttatccgacagcaatgcacattgccacatataagaaagtggttgaagcaacgattccggccatcgaacgtttacaaaaaaccttagcggcgaaatccgaagaattcaaagatgtggtgaaaatcggccgtacgcacttaatggatgccaccccgttgacattgggtcaggaattcagcggttatgctgcacaattaagtttcggtttagcggcaatcaaaaataccttaccgcatttacgccaactggcattaggcggtacggcagtgggtaccggtttaaatacacctaaaggctatgatgtaaaagtagcggaatatatcgccaaattcaccggcttgccgtttattaccgccgaaaacaaatttgaagcattagcaacacatgacgctatcgttgaaactcacggcgcattaaaacaagttgcgatgtccttattcaaaattgcaaatgatatccgtttattggcttcaggtcctcgttctggtatcggtgaaattttaattcctgaaaacgaaccgggttcatccatcatgccgggtaaagttaatccgacccaatgcgaagcgatgacaatggttgccgcacaagtattaggtaacgataccactatttcatttgccggttcgcaaggtcatttcgaattgaacgtattcaaaccggttatggcggcaaatttcctgcaatccgctcaattaatcgcagatgtttgcatttctttcgacgagcactgtgcaagcggcattaaaccaaatacgccgcgcattcaacacttacttgaaagttcattaatgttagtgaccgcattaaatactcatatcggttatgaaaatgcggcgaaaattgcgaaaactgcgcacaaaaacggtacaacattacgtgaagaggctatcaacttaggtttagtgtccgccgaagatttcgataaatgggttcgtccggaagatatggttggcagcttaaaa

SEQ ID NO: 8 - protein, 464 aa, Actinobacillus succinogenes
MTFRIEKDTMGEVQVPADKYWAAQTERSRNNFKIGPAASMPHEIIEAFGYLKKAAAYANADLGVLPAEKRDLIAQACDEILARKLDDQFPLVIWQTGSGTQSNMNLNEVIANRAHVINGGKLGEKSIIHPNDDVNKSQSSNDTYPTAMHIATYKKVVEATIPAIERLQKTLAAKSEEFKDVVKIGRTHLMDATPLTLGQEFSGYAAQLSFGLAAIKNTLPHLRQLALGGTAVGTGLNTPKGYDVKVAEYIAKFTGLPFITAENKFEALATHDAIVETHGALKQVAMSLFKIANDIRLLASGPRSGIGEILIPENEPGSSIMPGKVNPTQCEAMTMVAAQVLGNDTTISFAGSQGHFELNVFKPVMAANFLQSAQLIADVCISFDEHCASGIKPNTPRIQHLLESSLMLVTALNTHIGYENAAKIAKTAHKNGTTLREEAINLGLVSAEDFDKWVRPEDMVGSLK

SEQ ID NO: 9 - DNA, 2310 bp, Actinobacillus succinogenes
  CDS (1)..(2310)
atggctcaattaactgaagctcaacaaaaagcatgggaaggattcgttcccggtgaatggcaggaaggcgtaaatttacgtgactttatccaaaaaaactatacaccgtacgaaggcgacgaatcattcttggctgatgcgactccggcgaccactgaattgtggaacagcgtgatggaaggcatcaaaatcgaaaacaaaacccatgcgccgttggattttgatgaacatactccgtcaaccatcacttcccacaaaccgggctatatcgacaaagatttggaaaaaatcgtcggtttgcaaactgacgcaccgttaaaacgtgcaatcatgccgttcggcgggattaaaatgatcaaaggttcttgtcaggtttacggccgtactttagatcctaaagtcgaattcattttcaccgaataccgtaaaacccataaccaaggtgtattcgacgtttatacgccggacattttacgttgccgtaaatcaggtgtgctgaccggtttaccggatgcttacggtcgtggtcgtattatcggtgactaccgtcgtttagcggtatacggtatcgattacttaatgaaagataaaaaagctcaattcgattcattgcaaccgcgtttagaagcgggtgaagacattcaggctactattcagttacgtgaagaaattgccgaacaacatcgtgcgttaggtaaaatcaaagaaatggcggcgtcttacggttatgacatttccggtcctgcaatgaatgcacaggaagctattcaatggacttacttcgcttatttagctgcggttaaatcacaaaacggtgcggcaatgtctttcggtcgtacttcaacatttttagatatctatatcgaacgtgacttaaaacgcggtttaatcacagaacaacaagcgcaagaattaatggaccacttaattatgaaattacgtatggttcgtttcttacgtacgcctgaatacgatcaattattctccggcgacccgatgtgggcaaccgaaaccatcgcagggatgggattagacggtcgtccgttggttactaaaaacagtttccgtgtattacatactttatacactatgggcacgtctccggaaccgaacttaacgattctttggtccgaacaattacctgaagcgtttaaacgtttctgtgcgaaagtttccatcgatacttcatccgtacaatacgaaaacgacgatttaatgcgtcctgacttcaacaacgatgattacgcaatcgcatgttgcgtatctccgatggttgtcggtaaacaaatgcagttcttcggtgctcgtgcgaacttagcgaaaactatgttatacgctatcaacggcggtatcgacgagaaaaacggtatgcaggtcggccctaaaacagcaccgatcactgatgaagtattaaacttcgatactgttatcgaacgtatggacagcttcatggattggttggcgactcaatacgtaaccgcattaaacatcatccacttcatgcacgataaatacgcttatgaagcggcattgatggctttccatgatcgtgacgtattccgtaccatggcttgcggtattgccggactgtctgttgcagctgactcattgtctgcaatcaaatacgcgaaagttaaaccgattcgcggcgatattaaagacaaagacggtaacgtcgtagcgtcaaatgtagcgattgacttcgaaatcgaaggcgaatatccgcaattcggtaacaacgatccgcgcgtcgatgatttagcggttgatttggttgaacgtttcatgaaaaaagttcaaactcacaaaacttaccgtaatgccgtaccgacacaatctatcctgactatcacttctaacgtggtatacggtaagaaaaccggtaatactccggacggtcgtcgcgcaggcgcaccattcggaccgggtgcaaacccaatgcacggtcgcgaccaaaaaggtgcggtggcatcattaacatctgtagctaaattgccgtttgcttacgcgaaagacggtatttcatatacgttctcaatcgtaccgaacgcgttaggtaaagatgatgatgcgcaaaaacgtaacctggcaggtttattggacggttacttccaccacgaagcgacagtggaaggcggtcagcacttaaatgtgaacgtattgaatcgtgaaacattgttagacgcaatcgaccatcctgaaaaatatccgcaattaaccattcgcgtttcaggttatgccgttcgttttaactcattaacccgcgaacagcaacaagacgttatcacccgtacattcactcaagcaatg

SEQ ID NO: 10 - protein, 770 aa, Actinobacillus succinogenes
MAQLTEAQQKAWEGFVPGEWQEGVNLRDFIQKNYTPYEGDESFLADATPATTELWNSVMEGIKIENKTHAPLDFDEHTPSTITSHKPGYIDKDLEKIVGLQTDAPLKRAIMPFGGIKMIKGSCQVYGRTLDPKVEFIFTEYRKTHNQGVFDVYTPDILRCRKSGVLTGLPDAYGRGRIIGDYRRLAVYGIDYLMKDKKAQFDSLQPRLEAGEDIQATIQLREEIAEQHRALGKIKEMAASYGYDISGPAMNAQEAIQWTYFAYLAAVKSQNGAAMSFGRTSTFLDIYIERDLKRGLITEQQAQELMDHLIMKLRMVRFLRTPEYDQLFSGDPMWATETIAGMGLDGRPLVTKNSFRVLHTLYTMGTSPEPNLTILWSEQLPEAFKRFCAKVSIDTSSVQYENDDLMRPDFNNDDYAIACCVSPMVVGKQMQFFGARANLAKTMLYAINGGIDEKNGMQVGPKTAPITDEVLNFDTVIERMDSFMDWLATQYVTALNIIHFMHDKYAYEAALMAFHDRDVFRTMACGIAGLSVAADSLSAIKYAKVKPIRGDIKDKDGNVVASNVAIDFEIEGEYPQFGNNDPRVDDLAVDLVERFMKKVQTHKTYRNAVPTQSILTITSNVVYGKKTGNTPDGRRAGAPFGPGANPMHGRDQKGAVASLTSVAKLPFAYAKDGISYTFSIVPNALGKDDDAQKRNLAGLLDGYFHHEATVEGGQHLNVNVLNRETLLDAIDHPEKYPQLTIRVSGYAVRFNSLTREQQQDVITRTFTQAM

SEQ ID NO: 11 - DNA, 1212 bp, Actinobacillus succinogenes
  CDS (1)..(1212)
atgtccaaattagttttaattcttaactgcggtagctcatctttaaaatttgctatcttagatcccgtatctggtgaagaaaaattatcaggtttagccgaagctttctatcttccggaagcgcgtatcaaatggaaattgcacggtgaaaaaggcaatgcggatttaggtgccggagctgctcacagtgaagcgttaaactttattgttaaaaatattttccctttagacccttctttgcaggaaagtatcgtagcaatcggtcaccgtatcgttcacggcggtgagaaatttacctcttccgttatcgtcaccgacgaagtagttaaaggtatcgaagacgctattcaattcgcaccgttgcacaaccctgcccacttaatcggtataaaagaagcgttcaaaatcttcccgcatctgaaagataaaaacgtagtcgttttcgacaccgcattccatcaaaccatgcctgaagaagcattcttatacgctcttccttattctttatataaagaacacggtattcgtcgttacggcgcacacggtaccagccactattttatcagccgtgaagcggcaaaacgtttaggtgtcgcggaagataaaattaacgtgattacctgccacttaggtaacggcggttccgtttccgcaatccgccacggtgaatgtatcgatacctcaatgggcttaactccgttagaaggtatcgtaatgggtacccgttgcggcgacatcgatccggcgatcatgttctatatgcatgatactttgggcatgtccgtagaagaaatcaacacgaccttaaccaaaaaatccggtatcttaggtttaacggaagtgaccagtgactgtcgctttgcggaagacaactatgaaagtgatgacgaatcgttacgtgtaccggctcgtcgcgcaatggacgtttactgctaccgtttagcgaaatacatcggttcttatatggcggttatcggtgagcgtttagacgccatcgtgtttaccggcggtatcggtgaaaactccgctcacgtacgtgaaatcacgttgaatcacttaaaactgttcggttatcaattagatcgcgacaaaaatctggcggcccgtttcggtaacgaaggcgttatcacggcggataacacgccaatcgcaatggtaatcccgacaaatgaagaattggttatcgcacaggataccgcccgtctttgtttc

SEQ ID NO: 12 - protein, 404 aa, Actinobacillus succinogenes
MSKLVLILNCGSSSLKFAILDPVSGEEKLSGLAEAFYLPEARIKWKLHGEKGNADLGAGAAHSEALNFIVKNIFPLDPSLQESIVAIGHRIVHGGEKFTSSVIVTDEVVKGIEDAIQFAPLHNPAHLIGIKEAFKIFPHLKDKNVVVFDTAFHQTMPEEAFLYALPYSLYKEHGIRRYGAHGTSHYFISREAAKRLGVAEDKINVITCHLGNGGSVSAIRHGECIDTSMGLTPLEGIVMGTRCGDIDPAIMFYMHDTLGMSVEEINTTLTKKSGILGLTEVTSDCRFAEDNYESDDESLRVPARRAMDVYCYRLAKYIGSYMAVIGERLDAIVFTGGIGENSAHVREITLNHLKLFGYQLDRDKNLAARFGNEGVITADNTPIAMVIPTNEELVIAQDTARLCF

SEQ ID NO: 13 - DNA, 1437 bp, Actinobacillus succinogenes
  CDS (1)..(1437)
atggcgagaacaaactcatacgtgattggcgtgacgctggtcgctaccttaggcggacttttattcggttacgatacggctgtgatttcaggcacggtatcatccttagatacggtgtttatccaacctaaaggcttacctgaaatttcagcgaattcattgttggggttttgtgttgccagtgcgctgattggctgtatcatcggcggagcgtgcggcggctacttgagcagtaaatacggacgcaaaaaagccttagtgattgcggcggtattatttttgctttcagcattcggttccgcttatcccgaattcggtttaaccgaaataaaccgaaccaacgatgtaccttattatctcagcaacttcctcacccaatttgtgatttaccgtatcatcggcggcattggggttggaatcgcttccatgatttcaccgatgtatatcgcagaaatcgttccggcacatattcgcggcaaaatggtgtccttcaaccagtttgccatcattgcgggccaactcgtggtgtatttcgtaaattattttattgcgttaaacggtgataatacatggcttaatatgctgggttggcgctatatgtttttatcagaaatggtaccggccggattatttctaacgcttcttttctttgtaccggaaagtccgcgttggttagtattacaaaataaactcacacaagccgaagtcactttattaaaattattaggtgaaaaatcaggtaaaaaagaattacaaaacattgtttcttcgttagaacatcgtgtggcgaaaggcagcccgttgctgtccttcggtattggtattatcctaatcggcattgcactttccgtttttcagcaatttgtcggaatcaatgtggcgctttattatgcccctgagatttttaaatctttaggcgcaagtacggacagcgcattgttacaaaccattatcatgggggcgatcaacctttcttgtaccactattgctatttttactgtagataaatatggtcgtaaaccattacaaattgtcggtgcattaggtatggcggtaggcatgtgcgtattaggcaccgccttctacgctaatctttccggttcgattgcattaaccggcatgttattctatgtagcctcttttgccatctcttggggaccggtttgctgggtattattagccgaaattttcccgaatgcgattcgcagtcaagccttggcaatcgccgtagcaacacaatggattgcgaattatattgtttcctggacatttccgatgatggataaaagttcgtatttacttgaacattttaaccacggtttcgcctactgggtttatgcttttatgagtattttagctgcgttatttatgtggaaattcgtacctgaaaccaaaggcagaacattggaagaggtggaattattatggcgtaaaaaa

SEQ ID NO: 14 - protein, 479 aa, Actinobacillus succinogenes
MARTNSYVIGVTLVATLGGLLFGYDTAVISGTVSSLDTVFIQPKGLPEISANSLLGFCVASALIGCIIGGACGGYLSSKYGRKKALVIAAVLFLLSAFGSAYPEFGLTEINRTNDVPYYLSNFLTQFVIYRIIGGIGVGIASMISPMYIAEIVPAHIRGKMVSFNQFAIIAGQLVVYFVNYFIALNGDNTWLNMLGWRYMFLSEMVPAGLFLTLLFFVPESPRWLVLQNKLTQAEVTLLKLLGEKSGKKELQNIVSSLEHRVAKGSPLLSFGIGIILIGIALSVFQQFVGINVALYYAPEIFKSLGASTDSALLQTIIMGAINLSCTTIAIFTVDKYGRKPLQIVGALGMAVGMCVLGTAFYANLSGSIALTGMLFYVASFAISWGPVCWVLLAEIFPNAIRSQALAIAVATQWIANYIVSWTFPMMDKSSYLLEHFNHGFAYWVYAFMSILAALFMWKFVPETKGRTLEEVELLWRKK

SEQ ID NO: 15 - DNA, 1452 bp, Actinobacillus succinogenes
  CDS (1)..(1452)
atgtcagaaaaaggcgacatcggcgttatcggcttagcggtaatgggtcagaacttaattttaaatatgaacgacaacggctttaaagtcgtggcgttcaaccgtaccacctcaaaagtggatgaatttttacaaggtgcggcgaaaggcacaaacattatcggtgcttattctctggaagatttagccgcgaaattggaaaaaccgcgcaaagtaatgttaatggtgcgtgccggtgatgtggtggatcattttatcgatgctttattaccgcatttggaagcgggcgacatcattatcgacggcggtaactcgaattatccggatactaaccgccgcacacaagcgttggcggaaaaaggtgttcgctttatcggcaccggcgtttccggcggtgaagaaggcgcgcgtcacggaccttccatcatgccgggcggtaatcctgaagcctggccttatgtgaaacctattttgcaagcgatttccgccaaaacggacaaaggcgaaccttgctgcgattgggtgggcaaagaaggcgccggtcacttcgtgaaaatggtacataacggtatcgaatacggcgatatgcagctcatctgcgaagcttaccaattcttaaaagaaggtttgggtttaagttacgaagaaatgcatgaaatcttcaaacaatggaaaacaaccgaactcgacagctatctggtggacattaccaccgatattttggcatacaaagatacggacggtcagccgctggtggaaaaaatcttagacaccgccgggcaaaaaggtaccggtaaatggacggggatcaacgcgctagacttcggtattccgttgacattgattaccgaatccgtattcgcccgttgcgtatcttcattcaaacaacaacgagtcgaagcgtcaaaattattcaacaaaaccgtttcgccggttgaaggcgataaaaaagtgtggattgaagcggtacgcaaagccttgctcgcctccaaaatcatttcctacgcccaaggcttcatgttaatccgtgaagcatccgagcaattcgactggaacatcaactacggcgcgaccgctcttttatggcgcgaaggctgtatcatccgcagtgcatttttaggcaacattcgcgatgcttatgaagcgaacccggatttagtgttcctaggttccgacccgtatttcaaaggcattttagaaaatgctatggcggattggcgtaaagtggtggcgaagtccgtcgaagtgggcattccgatgccttgtatggcaagcgcgattactttcttagacggttacacttccgaacgcgtgccggcgaatttattgcaagcgcaacgcgactacttcggcgcacacacttacgaacgcaccgacaaaccgcgcggcgaattcttccacactaactggacgggtcgcggcggtaacacggcttccaccacttatgatgtg

SEQ ID NO: 16 - protein, 484 aa, Actinobacillus succinogenes
MSEKGDIGVIGLAVMGQNLILNMNDNGFKVVAFNRTTSKVDEFLQGAAKGTNIIGAYSLEDLAAKLEKPRKVMLMVRAGDVVDHFIDALLPHLEAGDIIIDGGNSNYPDTNRRTQALAEKGVRFIGTGVSGGEEGARHGPSIMPGGNPEAWPYVKPILQAISAKTDKGEPCCDWVGKEGAGHFVKMVHNGIEYGDMQLICEAYQFLKEGLGLSYEEMHEIFKQWKTTELDSYLVDITTDILAYKDTDGQPLVEKILDTAGQKGTGKWTGINALDFGIPLTLITESVFARCVSSFKQQRVEASKLFNKTVSPVEGDKKVWIEAVRKALLASKIISYAQGFMLIREASEQFDWNINYGATALLWREGCIIRSAFLGNIRDAYEANPDLVFLGSDPYFKGILENAMADWRKVVAKSVEVGIPMPCMASAITFLDGYTSERVPANLLQAQRDYFGAHTYERTDKPRGEFFHTNWTGRGGNTASTTYDV

SEQ ID NO: 17 - DNA, 32 bp, Artificial Sequence
  PCR Primer
cgttaaccgtgggaatcagtttgttaggaatg

SEQ ID NO: 18 - DNA, 53 bp, Artificial Sequence
  PCR Primer
cgaagttattgtaatacttccttttgctagtattgataatgaaatcctgtaag

SEQ ID NO: 19 - DNA, 58 bp, Artificial Sequence
  PCR Primer
ccataacttcgtataatgtatgctatacgaagttatttggggtaacgtaataaaaatg

SEQ ID NO: 20 - DNA, 31 bp, Artificial Sequence
  PCR Primer
tctctccttcgcggaataaaatatccacttc

SEQ ID NO: 21 - DNA, 82 bp, Artificial Sequence
  PCR Primer
ctagcaaaaggaagtattacaataacttcgtataatgtatgctatacgaagttataattcttgaagacgaaagggcctcgtg

SEQ ID NO: 22 - DNA, 57 bp, Artificial Sequence
  PCR Primer
cgtatagcatacattatacgaagttatggggtctgacgctcagtggaacgaaaactc

SEQ ID NO: 23 - DNA, 25 bp, Artificial Sequence
  PCR Primer
gcagcaatagaggaaacacggtttg

SEQ ID NO: 24 - DNA, 34 bp, Artificial Sequence
  PCR Primer
ggatttggtaccgtgccggcggcctaataacctg

SEQ ID NO: 25 - DNA, 30 bp, Artificial Sequence
  PCR Primer
cgaaccgaagcgttcctgcgcgagtaacgc

SEQ ID NO: 26 - DNA, 23 bp, Artificial Sequence
  PCR Primer
gcttcccattaatcaaacggcgg

SEQ ID NO: 27 - DNA, 31 bp, Artificial Sequence
  PCR Primer
ctcaaacaaaccgtgtttcctctattgctgc

SEQ ID NO: 28 - DNA, 46 bp, Artificial Sequence
  PCR Primer
aattatcaatgaggtgaagtatgacatttcgtattgaaaaagacac

SEQ ID NO: 29 - DNA, 56 bp, Artificial Sequence
  PCR Primer
gaattcgagctcggtacccggggatccctgaccgtcttcggtgaatactgatatag

SEQ ID NO: 30 - DNA, 35 bp, Artificial Sequence
  PCR Primer
acttcacctcattgataatttaaaattaaaaatcc

SEQ ID NO: 31 - DNA, 31 bp, Artificial Sequence
  PCR Primer
ggatccccgggtaccgagctcgaattcactg

SEQ ID NO: 32 - DNA, 30 bp, Artificial Sequence
  PCR Primer
cggtaccgagctcgaattcactggccgtcg

SEQ ID NO: 33 - DNA, 31 bp, Artificial Sequence
  PCR Primer
gtcatcttaacaggttattaggccgccggca

SEQ ID NO: 34 - DNA, 44 bp, Artificial Sequence
  PCR Primer
ccttcggccggccctgccgtttcggaaaactcacgctttacccg

SEQ ID NO: 35 - DNA, 42 bp, Artificial Sequence
  PCR Primer
ccttcggccggcccataaagaatccaagataaacgaattggc

(19) United States
(12) Patent Application Publication
(10) Pub. No.: US 2017/0306363 A1
(43) Pub. Date: Oct. 26, 2017

(54) METABOLIC ENGINEERING FOR ENHANCED SUCCINIC ACID BIOSYNTHESIS

(71) Applicant: Alliance for Sustainable Energy, LLC, Golden, CO (US)

(72) Inventors: Michael T. GUARNIERI, Denver, CO (US); Yat-Chen CHOU, Lakewood, CO (US); Gregg Tyler BECKHAM, Golden, CO (US); Davinia SALVACHÚA RODRÍGUEZ, Golden, CO (US)

(21) Appl. No.: 15/496,944

(22) Filed: Apr. 25, 2017

Related U.S. Application Data
(60) Provisional application No. 62/326,895, filed on Apr. 25, 2016.

(51) Int. Cl. C12P 7/46; C12N 9/88; C12N 9/10; C12N 9/04

(52) U.S. Cl. CPC C12P 7/46; C12Y 402/01002; C12Y 401/01032; C12Y 203/01054; C12N 9/0006; C12N 9/88; C12N 9/1029; C12Y 101/01037

(57) ABSTRACT

Presented herein are biocatalysts and methods for the production of succinic acid from carbon sources. The biocatalysts include microbial cells that have been engineered to overexpress exogenously added genes that encode enzymes active in the reductive branch of the tricarboxylic acid (TCA) cycle.

### CROSS-REFERENCE TO RELATED APPLICATION

This application claims priority to U.S. Provisional Application No. 62/326,895, filed Apr. 25, 2016, the contents of which are incorporated by reference in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### REFERENCE TO SEQUENCE LISTING

The instant application contains a Sequence Listing which has been submitted via EFS-web and is hereby incorporated by reference in its entirety. The ASCII copy, created on Apr. 24, 2017, is named 16-53_ST25.txt, and is 95,228 bytes in size.

### BACKGROUND

Biocatalytic production of commodity and specialty chemicals from renewable resources offers a high-potential, sustainable route to establish a viable bio-based economy. Recent advances in metabolic engineering and fermentation optimization have enabled the establishment of bio-based routes for the production of an array of biochemicals, fuel precursors, and pharmaceuticals. Four-carbon dicarboxylic acids are particularly promising precursors for the development of commodity and specialty chemicals. One such di-acid, succinic acid (SA), possesses properties analogous to petrochemically-derived maleic anhydride, a primary precursor to 1,4-butanediol (BDO), unsaturated polyester resins (UPR), lubricating oil additives, and an array of pharmaceutical and nutraceutical products.

The foregoing examples of the related art and limitations related therewith are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods that are meant to be exemplary and illustrative, not limiting in scope. In various embodiments, one or more of the above-described problems have been reduced or eliminated, while other embodiments are directed to other improvements.

Exemplary embodiments provide engineered cells with at least one exogenously added gene encoding an enzyme from the reductive branch of the tricarboxylic acid (TCA) cycle, where the cells are able to produce succinic acid from a carbon source.

In some embodiments, the exogenously added gene encodes malate dehydrogenase, PEP-carboxykinase, fumarase, or combinations thereof. In some embodiments, the cells contain an additional exogenously added gene encoding XylE or phosphoglucose dehydrogenase.

In certain embodiments, the cells are bacterial cells, such as bacterial cells from the genus Actinobacillus, from Actinobacillus succinogenes, or from Actinobacillus succinogenes strain 130Z.

In various embodiments, the cells produce a higher amount of succinic acid than the wild type cells.

In some embodiments, the carbon source is derived from lignocellulosic biomass and/or comprises glucose, xylose, galactose or arabinose.

In certain embodiments, the cells have an additional genetic modification that reduces the production of acetate or formate by the cell or reduces the expression of pyruvate formate lyase or acetate kinase.

Additional embodiments provide methods for producing succinic acid by culturing the engineered cells with a carbon source and recovering the succinic acid from the culture.

In some embodiments, the methods use carbon sources derived from lignocellulosic biomass.

In certain embodiments, the methods use bacterial cells from Actinobacillus succinogenes, cells that contain an exogenously added gene encoding malate dehydrogenase, or cells that have a genetic modification that reduces the expression of pyruvate formate lyase or acetate kinase.

In addition to the exemplary aspects and embodiments described above, further aspects and embodiments will become apparent by reference to the drawings and by study of the following descriptions.

### BRIEF DESCRIPTION OF THE DRAWINGS

Exemplary embodiments are illustrated in referenced figures of the drawings. It is intended that the embodiments and figures disclosed herein are to be considered illustrative rather than limiting.

FIG. 1 shows metabolic pathways and genetic modifications in A. succinogenes. Light grey arrows indicate native flux from pentose and hexose sugars. Medium grey arrows (e.g., PCK, MDH, FUM) indicate exemplary overexpression targets. Dark grey arrows (e.g., pflB, ackA) indicate exemplary genetic knockout targets. Table 1 lists the abbreviations used.

FIG. 2 shows an exemplary cassette for knocking out pflB (top) and an exemplary plasmid for overexpression of succinic acid pathway genes (bottom).

FIG. 3 shows different fermentation and metabolic parameters in wild-type A. succinogenes 130Z compared to the knockout strains ΔackA, ΔpflB, and ΔpflBΔackA. Scatter plots present (A) bacterial growth, utilization of (B) xylose and (C) glucose, and acid production: (D) SA, (E) acetic acid (AA), (F) formic acid (FA), (G) pyruvic acid (PA), and (H) lactic acid (LA). The bar graphs show (I) SA titers, (J) yields and metabolic yields, and (K) overall and maximum instantaneous productivities.

FIG. 4 shows different fermentation and metabolic parameters in wild-type A. succinogenes (130Z) compared to overexpression constructs pMDH and pPFM. The scatter plots show (A) bacterial growth, utilization of (B) xylose and (C) glucose, and acid production: (D) SA, (E) acetic acid (AA), (F) formic acid (FA), (G) pyruvic acid (PA), and (H) lactic acid (LA). The bar graphs show (I) SA titers, (J) yields and metabolic yields, and (K) overall and maximum instantaneous productivities.

FIG. 5 shows different fermentation and metabolic parameters in wild-type A. succinogenes (130Z) compared to ΔackA/pPMF, ΔpflB/pPMF, and ΔpflBΔackA/pPMF strains. The scatter plots show (A) bacterial growth, utilization of (B) xylose and (C) glucose, and acid production: (D) SA, (E) acetic acid (AA), (F) formic acid (FA), (G) pyruvic acid (PA), and (H) lactic acid (LA). The bar graphs show (I) SA titers, (J) yields and metabolic yields, and (K) overall and maximum instantaneous productivities.

FIG. 6 shows different fermentation and metabolic parameters in (A) pPCK and (B) pFUM strains.

FIG. 7 shows different fermentation and metabolic parameters in (A) pGDH and (B) ΔpflBpGDH strains.

FIG. 8 shows carbon recovery (carbon mole percent) of wild type and engineered strains. Carbon recovery was calculated using the metabolic model to account for unmeasured CO₂.

FIG. 9 shows the estimated NADH production and consumption for engineered strains. Moles of NADH produced (or consumed) were estimated using the metabolic model for each of the observed products.

TABLE 1

| Abbreviation | Description |
|---|---|
| Metabolites | |
| 13DPG | 3-Phospho-D-glyceroyl phosphate |
| 2PG | D-Glycerate 2-phosphate |
| 3PG | 3-Phospho-D-glycerate |
| 6PGC | 6-Phospho-D-gluconate |
| AC | Acetate |
| ACALD | Acetaldehyde |
| ACCOA | Acetyl-CoA |
| ACTP | Acetyl phosphate |
| DHAP | Dihydroxyacetone phosphate |
| E4P | D-Erythrose 4-phosphate |
| ETOH | Ethanol |
| F6P | D-Fructose 6-phosphate |
| FDP | D-Fructose 1,6-bisphosphate |
| FOR | Formate |
| FUM | Fumarate |
| G3P | Glyceraldehyde 3-phosphate |
| G6P | D-Glucose 6-phosphate |
| GLC | D-Glucose |
| MAL | L-Malate |
| OAA | Oxaloacetate |
| PEP | Phosphoenolpyruvate |
| PYR | Pyruvate |
| R5P | Alpha-D-Ribose 5-phosphate |
| RU5P | D-Ribulose 5-phosphate |
| S7P | Sedoheptulose 7-phosphate |
| SUCC | Succinate |
| XU5P | D-Xylulose 5-phosphate |
| XYL | D-Xylose |
| XU | D-Xylulose |
| LAC | D-Lactate |
| Gene Targets | |
| pflB | Pyruvate formate lyase |
| ackA | Acetate kinase |
| PCK | Phosphoenolpyruvate carboxykinase |
| MDH | Malate dehydrogenase |
| FUM | Fumarase |

### DETAILED DESCRIPTION

Presented herein are biocatalysts and methods for the production of succinic acid from carbon sources. The biocatalysts include microbial cells that have been engineered to overexpress exogenously added genes that encode enzymes active in the reductive branch of the tricarboxylic acid (TCA) cycle. The cells may also include genetic modifications that ablate the expression of genes that encode enzymes involved in formate or acetate biosynthesis. The modifications allow the engineered cells to produce higher amounts of succinic acid or higher purity succinic acid in comparison to wild type cells.

Succinic acid is a key chemical intermediate and potential platform alternative to petro-derived maleic anhydride for the synthesis of an array of high-value industrial products. Succinic acid is a specialty chemical and an important precursor for the synthesis of high-value products that can be applied across many industries, such as biopolymers, pharmaceutical products, and foods.

Microbial production of commodity and specialty chemicals from renewable resources offers a promising, sustainable route to establish a viable bioeconomy. Four-carbon dicarboxylic acids represent interesting precursors for bioproducts. Succinic acid (SA) in particular exhibits properties analogous to petrochemically-derived maleic anhydride, a primary precursor to 1,4-butanediol (BDO), unsaturated polyester resins, lubricating oil additives, and an array of other products. The similarity of SA to maleic anhydride, coupled with its natural occurrence as a byproduct in microbial fermentation, has made it an attractive target chemical. SA also presents potential for a series of unique product suites, including the biodegradable polyester polybutylene succinate (PBS). Elucidation of mechanisms governing SA biosynthesis in relevant industrial hosts offers a means to develop strain engineering strategies aimed at flux enhancement.

At present, bio-based production of succinic acid is not competitive with petroleum-derived analogs, largely due to technical hurdles associated with low cost fermentation and product recovery. Biosynthetic enhancements to succinic acid titers, productivity rates, and carbon yields via rational metabolic and fermentation engineering strategies offer a targeted approach to address these hurdles. Further, reduction or elimination of heterofermentative co-products via additional strain-engineering approaches offers an additional means to enhance carbon yield, while concurrently increasing product purity, which will in turn improve product recovery (estimated to account for upwards of 60-70% of the overall bioprocess cost). Thus, development of economically viable biobased routes to succinic acid will require rational strain-engineering approaches targeting increased flux to succinic acid and elimination of competitive carbon pathways.

Development and deployment of anaerobic strains with high native flux to SA, such as Actinobacillus succinogenes 130Z, offers an appealing foundation for effective biocatalysts. A. succinogenes is a gram-negative, capnophilic, facultatively anaerobic, biofilm-forming bacterium with the capacity to convert a broad range of carbon sources to SA as a primary fermentative product, achieving among the highest reported SA titers and yields to date. Because A. succinogenes is capnophilic, it can incorporate CO₂ into SA, making this organism an ideal host for conversion of lignocellulosic sugars and CO₂ to an emerging commodity bioproduct sourced from renewable feedstocks.

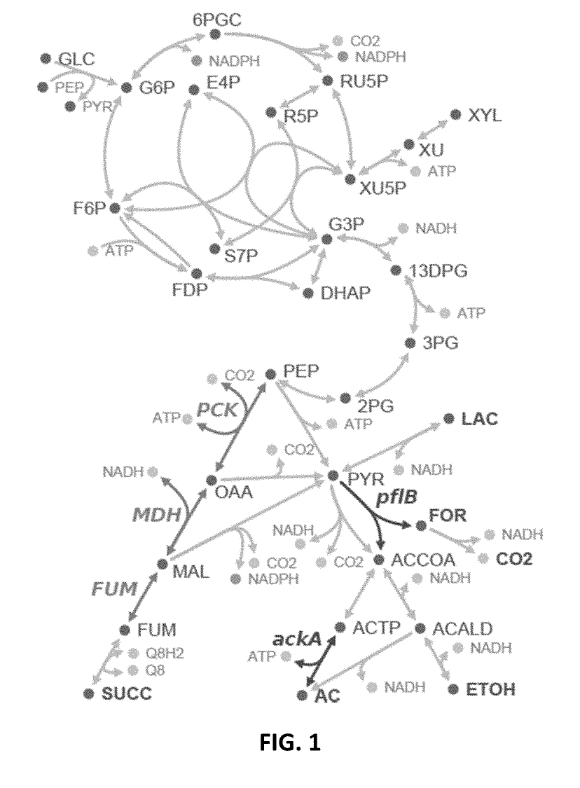

The core metabolic and SA biosynthetic pathways in A. succinogenes (and related organisms), including an incomplete TCA cycle which natively terminates at SA, are depicted in FIG. 1. Identification and manipulation of additional strain engineering targets offers a means to further enhance SA flux and resultant productivity. To date, successful metabolic engineering of A. succinogenes has been difficult, in part due to the organism's limited tractability, and thus, mechanisms governing flux to SA remain to be fully elucidated.

Disclosed herein are genetic tools to systematically manipulate competing acid production pathways and overexpress the succinic acid-producing machinery in organisms such as Actinobacillus succinogenes. These metabolic engineering capabilities enable examination of SA flux determinants via knockout of the primary competing pathways—namely acetate and formate production—and overexpression of enzymes in the reductive branch of the TCA cycle leading to SA. The genetic modifications disclosed herein can lead to succinic acid production improvements, and also allow the identification of key flux determinants and new bottlenecks and energetic needs when removing by-product pathways in microbial metabolism. The overexpression of the SA biosynthetic machinery, for example the enzyme malate dehydrogenase, enhances flux to SA. Additionally, removal of competitive carbon pathways leads to higher purity SA. The resultant engineered strains also lend insight into energetic and redox balance and elucidate mechanisms governing organic acid biosynthesis in this important natural SA-producing microbe.

The resulting strains are capable of fermentation on myriad carbon sources, including carbohydrate streams derived from lignocellulosic biomass. Examples include pentose-rich sugar streams from corn stover or culturing the cells in media with added sugars such as glucose, xylose, arabinose, galactose, or other sugars. Lignocellulose may be pretreated (e.g., with dilute acids) or contacted with enzymes such as cellulases to generate fermentable carbon sources.

Also disclosed are gene overexpression and/or marker-less gene knockout microbial strains, including strains of A. succinogenes. The knockout of competitive carbon pathway targets leads to higher-purity production of SA. Concurrently, up-regulation of the reductive branch of the TCA cycle enhances flux to SA, resulting in titer, rate, and yield enhancements. These modifications reveal a finely tuned energetic and redox system and previously unidentified mechanisms of secondary organic acid biosynthesis. Additionally, the resultant strains present promising metabolic engineering strategies for the economically viable, sustainable production of SA from pentose-rich sugar streams.

Disclosed herein are metabolic modeling techniques to identify strain-engineering targets and novel genetic tools to pursue rational metabolic engineering strategies coupled with fermentation optimization routes. Specific examples are provided below, but the content of this disclosure also includes overexpression and knockout strains (and combinations thereof) of microorganisms wherein the gene targets are part of the succinic acid pathway or alter the ability of a microorganism to produce succinic acid.

Gene knockout targets also include biosynthetic components governing acetate and formate biosynthesis, both in isolated and coupled background strains. Knockout of these targets leads to near homo-fermentative production of succinic acid. Concurrently, the reductive branch of the TCA cycle may be up-regulated via overexpression of PEP-carboxykinase (PEPCK), malate dehydrogenase (MDH), and fumarase (FUM). For example, overexpression of these three genes on an operon leads to significant enhancement of succinic acid titers, rates, and yields. Exemplary gene targets include PEP-carboxykinase, malate dehydrogenase, fumarase, pyruvate formate lyase, acetate kinase, malic enzyme, formate dehydrogenase, and others. Additional strain-engineering targets and combinations thereof are presented in Tables 2 and 3. Microorganisms may be engineered to overexpress any of the gene targets disclosed herein or sequences presented herein. Likewise, any of these genes or sequences may be ablated in a microorganism.

TABLE 2

| Strain or plasmid | Description |
|---|---|
| Strains | |
| Actinobacillus | |
| succinogenes | |
| 130Z | wild-type |
| ΔpflB::bla | 130Z derivative; contains loxP-bla-loxloxP integration |
| | replacing pflB; |
| ΔpflB | ΔpflB::bla derivative; contains one loxP site |
| ΔackA | 130Z derivative; contains ackA knockout |
| ΔpflBΔackA | 130Z derivative; contains pflB and ackA knockouts |
| Plasmids | |
| pLGZ920 | E. coli - A. succinogenes shuttle vector; Amp^{r}; |
| | contains PpckA |
| pL920Cm | pLGZ920 derivative; Cm^{r}; Cm^{r} gene under the control |
| | of Pbla |
| pPCK | pLGZ920 derivative; Amp^{r}; pckA under the control |
| | of PpckA |
| pMDH | pLGZ920 derivative; Amp^{r}; mdh under the control |
| | of Pmdh |
| pFUM | pLGZ920 derivative; Amp^{r}; fum under the control |
| | of PpckA |
| pPMF | pLGZ920 derivative; Cm^{r}; pckA under the control |
| | of PpckA; mdh under the control of Pmdh; fum under |
| | the control of Pfum |
| pLCreCm | pLGZ920 derivative; Cm^{r}; cre under the control of |
| | PpckA |

Phosphoenolpyruvate carboxykinase (PEP-carboxykinase or PEPCK) catalyzes the conversion of oxaloacetate to phosphoenolpyruvate and carbon dioxide. Nucleic acid and amino acid sequences for PEPCK from A. succinogenes are provided as SEQ ID NOS:1 and 2, respectively. Malate dehydrogenase (MDH) reversibly catalyzes the oxidation of malate to oxaloacetate using the reduction of NAD⁺ to NADH. Nucleic acid and amino acid sequences for two MDH isoforms from A. succinogenes are provided as SEQ ID NOS:3 and 4, respectively (MDH1) and SEQ ID NOS:5 and 6, respectively (MDH2). Fumarase (fumarate hydratase or FUM) catalyzes the reversible hydration/dehydration of fumarate to malate. Nucleic acid and amino acid sequences for FUM from A. succinogenes are provided as SEQ ID NOS:7 and 8, respectively.

Pyruvate formate lyase (PFL or pflB) catalyzes the reversible conversion of pyruvate and coenzyme-A into formic acid and acetyl-CoA. Nucleic acid and amino acid sequences for PFL from A. succinogenes are provided as SEQ ID NOS:9 and 10, respectively. Acetate kinase (AK or ackA) catalyzes conversion of acetyl-phosphate and ADP to acetate and ATP. Nucleic acid and amino acid sequences for AK from A. succinogenes are provided as SEQ ID NOS:11 and 12, respectively.

XylE is a transporter protein that facilitates cellular uptake and utilization of the sugar xylose. Nucleic acid and amino acid sequences for XylE from A. succinogenes are provided as SEQ ID NOS:13 and 14, respectively. Phosphogluconate dehydrogenase (GDH) is an enzyme in the pentose phosphate pathway that catalyzes the production of ribulose-5-phosphate from 6-phosphogluconate. Nucleic acid and amino acid sequences for GDH from A. succinogenes are provided as SEQ ID NOS:15 and 16, respectively.

Exemplary microorganisms suitable for genetic engineering as described herein include bacteria, including those from the genus Actinobacillus or strains of A. succinogenes such as A. succinogenes 130Z. Additional examples include the following Actinobacillus strains: A. actinomycetemcomitans, A. anseriformium, A. arthritidis, A. capsulatus, A. delphinicola, A. equuli, A. hominis, A. indolicus, A. hgnieresii, A. minor, A. muris, A. pleuropneumoniae, A. porcinus, A. rossii, A. scotiae, A. seminis, A. suis, and A. ureae. Bacteria may also include E. coli, Anaerobiospirillum succiniciproducens, Corynebacterium glutamicum or Mannheimia succiniciproducens.

Biocatalytic production of industrial platform chemicals from renewable substrates offers a promising means to generate petrochemical alternatives. Biological production of SA from lignocellulosic substrates in particular has the potential to displace a significant fraction of petroleum-derived maleic anhydride, serving as a potentially high-volume functional replacement in the production of an array of biopolymers and biomaterials. The development of biocatalysts with enhanced SA biosynthetic capacity via metabolic engineering strategies focused upon removal of alternative carbon sinks and/or enhanced biosynthetic pathway flux leads to strains that demonstrate a number of favorable characteristics, including enhanced titers, rates, and yields of SA, as well as reduction of alternative fermentative products, all of which will serve to enhance bioprocess economics.

Interplay exists between formic and acetic acid biosynthesis in redox and energetic balance, and biosynthesis of acetic acid may be a key contributor to SA productivity. Additionally, acetic acid biosynthetic capacity exists in A. succinogenes mutants lacking a canonical acetate kinase gene, indicating an alternative biosynthetic route. The genetic and/or flux modifications disclosed herein may also lead to increased co-production of lactic acid.

Modified microorganisms disclosed herein may be used in batch or continuous fermentation processes. Growth lags and delays in the onset of SA production may be mitigated by employing a continuous fermentation process. For example, a continuous process may benefit from deployment of the ΔpflB/pPMF combinatorial strain, wherein steady-state SA production is relatively unaffected compared to wild-type, yet heterofermentative products are significantly reduced, enabling more facile in-line separations.

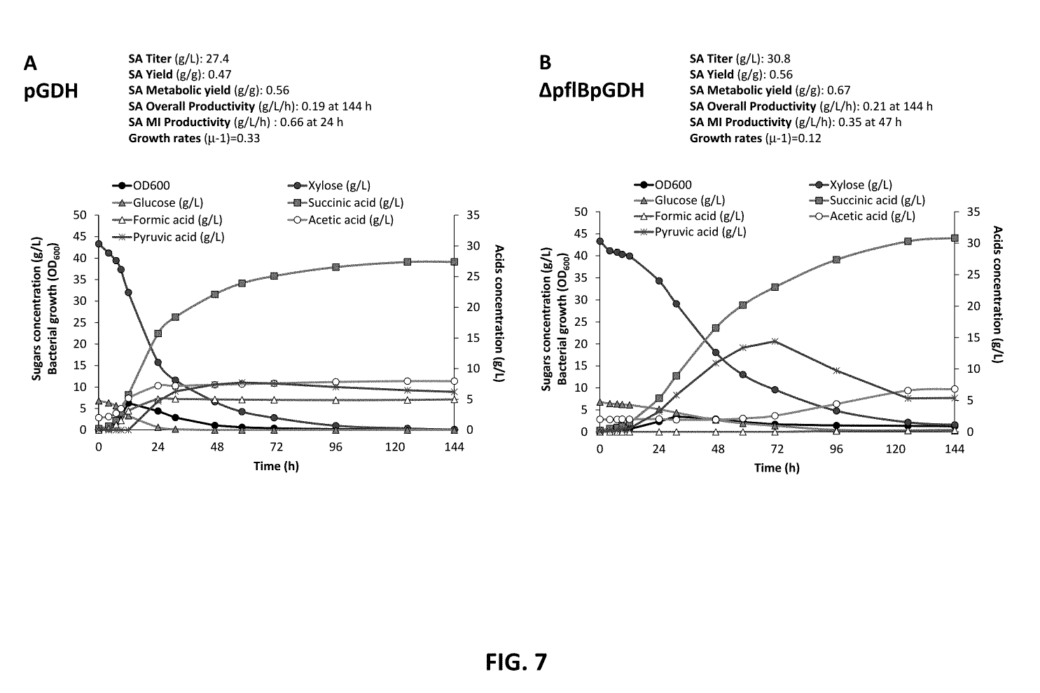

Additional strain modifications, including components of the pentose phosphate pathway, may also be used to increase upstream flux enhancement. For example, overexpression of phosphoglucose dehydrogenase (GDH) in wild-type and pflB mutant backgrounds may result in SA biosynthetic production enhancement (FIG. 7). Varying the carbon source employed may also lead to production improvements, either alone or in conjunction with engineering strains to express transporter proteins like XylE. Such modifications may increase utilization of specific sugars and also increase productivity. Additional targets include genes involved in the generation of reductant and/or ATP.

TABLE 3
Exemplary Gene Targets and Engineered Strains

| Gene Target(s) | Overexpression or KO |
|---|---|
| PEPCK | Overexpression |
| MDH | Overexpression |
| FUM | Overexpression |
| FUM, MDH | Overexpression |
| PEPCK, MDH, FUM | Overexpression |
| Malic Enzyme (ME) | Overexpression |
| ME, PEPCK, FUM | Overexpression |
| ME, PEPCK | Overexpression |
| AK | Overexpression |
| XYLE | Overexpression |
| FDH | Overexpression |
| ArcAB | Overexpression |
| AK | Knockout |
| PFL | Knockout |
| PFL/AK | Double Knockout |

In certain embodiments, a nucleic acid may be identical to the sequence represented herein. In other embodiments, the nucleic acids may be least about 80%, 81%, 82%, 83%, 84%, 85%, 86%, 87%, 88%, 89%, 90%, 91%, 92%, 93%, 94%, 95%, 96%, 97%, 98% or 99% identical to a nucleic acid sequence presented herein, or 80%, 81%, 82%, 83%, 84%, 85%, 86%, 87%, 88%, 89%, 90%, 91%, 92%, 93%, 94%, 95%, 96%, 97%, 98% or 99% identical to a nucleic acid sequence presented herein. Sequence identity calculations can be performed using computer programs, hybridization methods, or calculations. Exemplary computer program methods to determine identity and similarity between two sequences include, but are not limited to, the GCG program package, BLASTN, BLASTX, TBLASTX, and FASTA. The BLAST programs are publicly available from NCBI and other sources. For example, nucleotide sequence identity can be determined by comparing query sequences to sequences in publicly available sequence databases (NCBI) using the BLASTN2 algorithm.

The nucleic acid molecules exemplified herein encode polypeptides with amino acid sequences represented herein. In certain embodiments, the polypeptides may be at least about 80%, 81%, 82%, 83%, 84%, 85%, 86%, 87%, 88%, 89%, 90%, 91%, 92%, 93%, 94%, 95%, 96%, 97%, 98% or 99% identical to the reference amino acid sequence while possessing the enzymatic function. The present disclosure encompasses bacterial cells such as A. succinogenes cells that contain the nucleic acid molecules described herein, have genetic modifications to the nucleic acid molecules, or express the polypeptides described herein.

“Nucleic acid” or “polynucleotide” as used herein refers to purine- and pyrimidine-containing polymers of any length, either polyribonucleotides or polydeoxyribonucleotide or mixed polyribo-polydeoxyribonucleotides. This includes single- and double-stranded molecules (i.e., DNA-DNA, DNA-RNA and RNA-RNA hybrids) as well as “protein nucleic acids” (PNA) formed by conjugating bases to an amino acid backbone. This also includes nucleic acids containing modified bases.

Nucleic acids referred to herein as “isolated” are nucleic acids that have been removed from their natural milieu or separated away from the nucleic acids of the genomic DNA or cellular RNA of their source of origin (e.g., as it exists in cells or in a mixture of nucleic acids such as a library), and may have undergone further processing. Isolated nucleic acids include nucleic acids obtained by methods described herein, similar methods or other suitable methods, including essentially pure nucleic acids, nucleic acids produced by chemical synthesis, by combinations of biological and chemical methods, and recombinant nucleic acids that are isolated. In certain embodiments, the nucleic acids are complementary DNA (cDNA) molecules.

The nucleic acids described herein may be used in methods for production of organic acids (e.g., succinic acid) through incorporation into cells, tissues, or organisms. In some embodiments, a nucleic acid may be incorporated into a vector for expression in suitable host cells. Alternatively, gene-targeting or gene-deletion vectors may also be used to disrupt or ablate a gene. The vector may then be introduced into one or more host cells by any method known in the art. One method to produce an encoded protein includes transforming a host cell with one or more recombinant nucleic acids (such as expression vectors) to form a recombinant cell. The term “transformation” is generally used herein to refer to any method by which an exogenous nucleic acid molecule (i.e., a recombinant nucleic acid molecule) can be inserted into a cell, but can be used interchangeably with the term “transfection.”

Suitable vectors for gene expression may include (or may be derived from) plasmid vectors that are well known in the art, such as those commonly available from commercial sources. Vectors can contain one or more replication and inheritance systems for cloning or expression, one or more markers for selection in the host, and one or more expression cassettes. The inserted coding sequences can be synthesized by standard methods, isolated from natural sources, or prepared as hybrids. Ligation of the coding sequences to transcriptional regulatory elements or to other amino acid encoding sequences can be carried out using established methods. A large number of vectors, including algal, bacterial, yeast, and mammalian vectors, have been described for replication and/or expression in various host cells or cell-free systems, and may be used with genes encoding the enzymes described herein for simple cloning or protein expression.

Certain embodiments may employ promoters or regulatory operons. The efficiency of expression may be enhanced by the inclusion of enhancers that are appropriate for the particular cell system that is used, such as those described in the literature. Suitable promoters also include inducible promoters. Expression systems for constitutive expression in bacterial cells are available from commercial sources. Inducible expression systems are also suitable for use.

Host cells can be transformed, transfected, or infected as appropriate with gene-disrupting constructs or plasmids (e.g., an expression plasmid) by any suitable method including electroporation, calcium chloride-, lithium chloride-, lithium acetate/polyethylene glycol-, calcium phosphate-, DEAE-dextran-, liposome-mediated DNA uptake, spheroplasting, injection, microinjection, microprojectile bombardment, phage infection, viral infection, or other established methods. Alternatively, vectors containing a nucleic acid of interest can be transcribed in vitro, and the resulting RNA introduced into the host cell by well-known methods, for example, by injection. Exemplary embodiments include a host cell or population of cells expressing one or more nucleic acid molecules or expression vectors described herein (for example, a genetically modified microorganism). The cells into which nucleic acids have been introduced as described above also include the progeny of such cells.

Vectors may be introduced into host cells by direct transformation, in which DNA is mixed with the cells and taken up without any additional manipulation, by conjugation, electroporation, or other means known in the art. Expression vectors may be expressed by host cells episomally or the gene of interest may be inserted into the chromosome of the host cell to produce cells that stably express the gene with or without the need for selective pressure. For example, expression cassettes may be targeted to neutral chromosomal sites by double recombination.

Host cells with targeted gene disruptions or carrying an expression vector (i.e., transformants or clones) may be selected using markers depending on the mode of the vector construction. The marker may be on the same or a different DNA molecule. In prokaryotic hosts, the transformant may be selected, for example, by resistance to ampicillin, tetracycline or other antibiotics. Production of a particular product based on temperature sensitivity may also serve as an appropriate marker.

In exemplary embodiments, the host cell may be a microbial cell, such as a bacterial cell, and may be from any genera or species of bacteria that is known to be genetically manipulable. Exemplary microorganisms include, but are not limited to, bacteria; fungi; archaea; protists; eukaryotes, such as algae; and animals such as plankton, planarian, and amoeba.

Host cells may be cultured in an appropriate fermentation medium. An appropriate, or effective, fermentation medium refers to any medium in which a host cell, including a genetically modified microorganism, when cultured, is capable of growing and/or expresing recombinant proteins. Such a medium is typically an aqueous medium comprising assimilable carbon, nitrogen and phosphate sources, but can also include appropriate salts, minerals, metals and other nutrients. Microorganisms and other cells can be cultured in conventional fermentation bioreactors or photobioreactors and by any fermentation process, including batch, fed-batch, cell recycle, and continuous fermentation. The pH of the fermentation medium is regulated to a pH suitable for growth of the particular organism. Culture media and conditions for various host cells are known in the art. A wide range of media for culturing bacterial cells, for example, are available from ATCC.

Isolation or extraction of succinic acid from the cells may be aided by mechanical processes such as crushing, for example, with an expeller or press, by supercritical fluid extraction, pH-induced precipitation, or the like. Once the succinic acid has been released from the cells, it can be recovered or separated from a slurry of debris material (such as cellular residue, by-products, etc.). This can be done, for example, using techniques such as sedimentation or centrifugation. Recovered succinic acid can be collected and directed to a conversion process if desired.

Processes for producing succinic acid may also comprise a removing step, wherein a portion of the succinic acid is removed from the mixture to form substantially pure succinic acid. In some embodiments, the removing step may comprise a two-step process, wherein the first step comprises an adsorption process wherein the succinic acid is not adsorbed, and other components are selectively adsorbed. In some embodiments of the present invention, a first step comprising adsorption will result in a substantially pure succinic acid stream.

The first step may be followed by a second polishing step. The second step may comprise crystallization. In still further embodiments of the present invention, the removing step is at least one of affinity chromatography, ion exchange chromatography, solvent extraction, liquid-liquid extraction, distillation, filtration, centrifugation, electrophoresis, hydrophobic interaction chromatography, gel filtration chromatography, reverse phase chromatography, chromatofocusing, differential solubilization, preparative disc-gel electrophoresis, isoelectric focusing, HPLC, reversed-phase HPLC, or countercurrent distribution, or combinations thereof. In some embodiments of the present invention, the removing step may be performed either during the mixing step or subsequent to the mixing step or both.

Separation/purification operations may be used to generate succinic acid free of or substantially free of a wide variety of impurities that may be introduced during the biological production of the succinic acid. These impurities may include fermentation salts, nutrients and media to support growth, unconverted substrate, extracellular proteins and lysed cell contents, as well as the buildup of non-target metabolites. Accumulation of these constituents in culture broth may vary greatly depending on the microorganism, substrate used for conversion, biological growth conditions and bioreactor design, and broth pretreatment.

Cell removal from the broth may be achieved by a variety of solid removal unit operations. Some examples include filtration, centrifugation, and combinations thereof. Once the microorganism cell matter has been removed, further impurity removal operations may be utilized such as exposing the mixture to activated carbon.

Examples of biomass and other lignocellulosic-containing materials that may be degraded to sugars for use as carbon sources include bioenergy crops, agricultural residues, municipal solid waste, industrial solid waste, sludge from paper manufacture, yard waste, wood, forestry waste, corn grain, corn cobs, crop residues such as corn husks, corn stover, corn fiber, grasses, wheat, wheat straw, barley, barley straw, hay, rice straw, switchgrass, waste paper, sugar cane bagasse, sorghum, soy, components obtained from milling of grains, trees, branches, roots, leaves, wood (e.g., poplar) chips, sawdust, shrubs and bushes, vegetables, fruits, flowers and animal manure.

Biomass or other lignocellulosic feedstocks may be subjected to pretreatment at an elevated temperature in the presence of a dilute acid, concentrated acid or dilute alkali solution for a time sufficient to at least partially hydrolyze the hemicellulose components before adding enzymes capable of producing carbohydrates such as sugars from the lignocellulose. Additional suitable pretreatment regimens include ammonia fiber expansion (AFEX), treatment with hot water or steam, or lime pretreatment. Following any pretreatment steps, lignocellulose may be subjected to saccharification by contacting the lignocellulose with enzymes such as cellulases, endoglucanases, β-glucosidases and other enzymes to hydrolyze the lignocellulose to sugars.

### EXAMPLES

### Example 1

Core-Carbon Metabolic Model of A. succinogenes 130Z

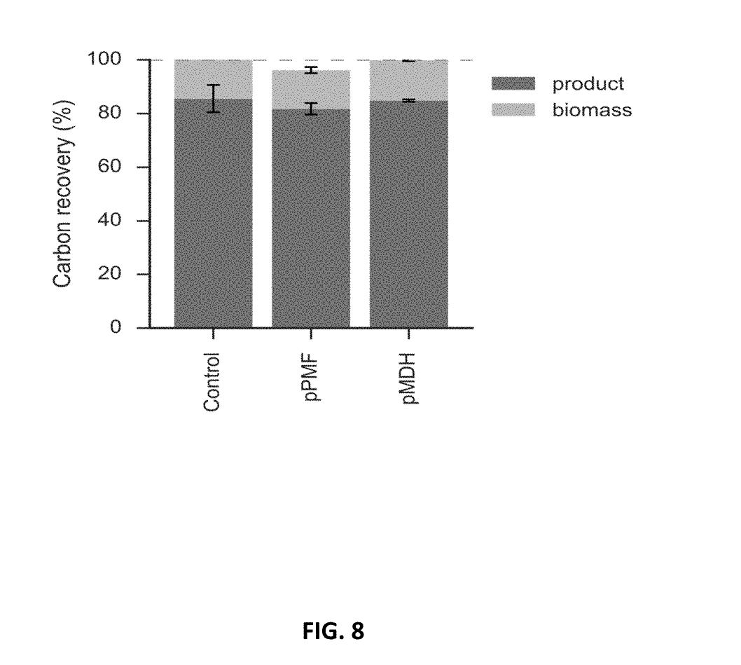

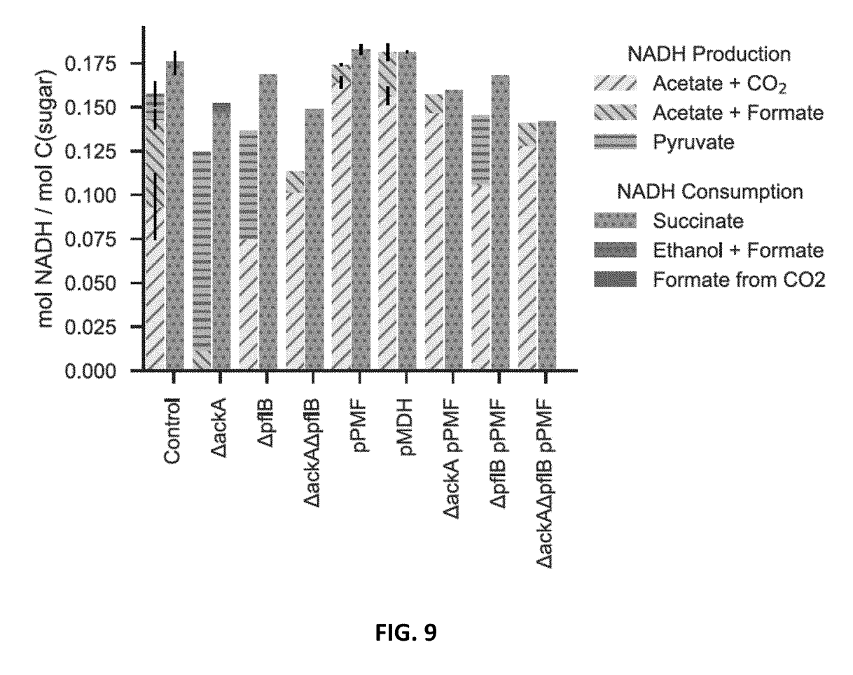

A core-carbon metabolic model of A. succinogenes 130Z was created following the genome annotation provided in McKinlay et al., BMC Genomics 11:680 (2010). The model consists of 51 intracellular metabolites, 20 extracellular metabolites, and 89 mass-balanced reactions. Pathways for the metabolism of glucose, xylose, galactose, and arabinose were included based on annotated pathways and stoichiometry from the MetaCyc database. The model was used to predict metabolite fluxes, including NADH and CO₂. By condensing the model to a number of balanced reactions that produce the observed fermentation products, the amount of CO₂ and NADH absorbed and consumed by the creation of each product was found. This accounting allowed the overall mass balance (FIG. 8) and redox balance (FIG. 9) to be calculated.

### Example 2

### Microorganisms and Growth Conditions

Actinobacillus succinogenes 130Z (ATCC 55618) was anaerobically cultivated in sterile capped bottles (100 mL) containing 50 mL of Tryptic Soy Broth (TSB) supplemented with 10 g/L glucose (TSBG; Fluka Analytical, India) and incubated overnight at 37° C. and 120 rpm. Cells were harvested by centrifugation (Sorvall) then resuspended in 5 mL TSB and 5 mL glycerol, aliquoted in cryovials, and stored at −70° C. Prior to the inoculum preparation, bacteria were revived from the glycerol stock at the same conditions detailed above. Bacterial growth was followed by optical density measurements at 600 nm (OD₆₀₀). Serum bottle cultures were inoculated at an OD₆₀₀ of 0.1 with plate-harvested biomass.

### Example 3

### Preparation of Fermentation Seed Culture and Fermentation Media

A. succinogenes strains were anaerobically grown in 150 mL sterile capped bottles containing 50 mL of TSBG (TSB+glucose) and antibiotic (30 mg/L chloramphenicol or 100 mg/L ampicillin, as indicated in Table 2) (excluding the control strain, 130Z), and incubated overnight at 37° C. and 200 rpm. Cells were inoculated in the fermenter at an initial OD₆₀₀ of 0.05. To ensure anaerobic fermentation, CO₂ was sparged overnight before bacterial inoculation.

The media used for fermentations consisted of 6 g yeast extract, 10 g corn steep liquor (Sigma-Aldrich, USA) (prepared as described below), 0.3 g Na₂HPO₄, 1.4 g NaH₂PO₄, 1.5 g K₂HPO₄, 1 g NaCl, 0.2 g MgCl₂×6H₂O, and 0.2 g CaCl₂×2H₂O. Corn steep liquor was prepared at a concentration of 200 g/L (20×) and then boiled at 105° C. for 15 minutes. After cooling, solids were separated and the supernatant was autoclaved and used as nutrient source. As a carbon source, a mixture of sugars mimicking the concentration in real biomass hydrolysates (in particular deacetylated and diluted acid pretreated hydrolysate (DDAPH) was utilized. The final sugar concentration was 60 g/L and consisted of 6.5 g/L glucose, 44 g/L xylose, 3.5 g/L galactose, and 6.5 g/L arabinose. In addition, acetic acid (1.7 g/L) was supplemented to the media since DDAPH contains this acid. Appropriate antibiotic was added to the fermentor prior to inoculation.

### Example 4

### Fermentation Conditions

Fermentations were performed in 0.5-L BioStat-Q Plus fermentors (Sartorious) with 300 mL of media. The pH was maintained at 6.8 by supplementing 4N NaOH. The temperature was controlled at 37° C. and the agitation at 300 rpm. During the fermentation, CO₂ was sparged at 0.1 vvm. Samples (about 1 mL) from the fermentations were taken in aseptic conditions at various time points to follow bacterial growth, sugar consumption, and the production or uptake of acids (e.g., SA, formic acid, acetic acid, lactic acid, pyruvic acid) or ethanol. All the fermentations were performed at least in duplicate.

### Example 5

### Analytical Methods

Bacterial growth was tracked by OD₆₀₀ in a spectrophotometer, as a measurement of cells in suspension in the fermentation broth. Growth rates (μ⁻¹) were calculated from the maximum growth slope before cell aggregation occurred. Samples were then filtered through a 0.2 μm syringe filter before placing them in high pressure liquid chromatography (HPLC) vials to analyze carbohydrates (glucose, xylose, arabinose, and galactose), organic acids (SA, formic acid, acetic acid, lactic acid, pyruvic acid), and ethanol. Carbohydrate HPLC analysis was performed by injecting 6.0 μL of 0.2 μm filtered culture supernatant onto an Agilent 1100 series system equipped with a Phenomenex Shodex SUGAR 7u SP0810 20A Column 300 mm×8 mm plus an anion guard column and cation guard column (Bio-Rad Laboratories) at 85° C. run using a mobile phase of Nanopure water at a flow rate of 0.6 mL/min and a refractive index detector for detection.

HPLC analyses for ethanol and all organic acids, except for pyruvic acid, were performed by injecting 6.0 μL of 0.2 μm filtered culture supernatant onto an Agilent 1100 series system equipped with a Bio-Rad HPLC Organic Acid Analysis Column, Aminex HPX-87H Ion Exclusion Column 300 mm×7.8 mm and a cation H⁺ guard column (Bio-Rad Laboratories) at 55° C. using a mobile phase of 0.01 N sulfuric acid at a flow rate of 0.6 mL/min and a refractive index detector for detection. Pyruvic acid HPLC analysis was performed by injecting 6.0 μL of 0.2 μm filtered culture supernatant onto an Agilent 1100 series system equipped with a Phenomenex Rezex RFQ-Fast Acid H⁺ (8%) Column 100 mm×7.8 mm and a cation guard cartridge (Bio-Rad Laboratories) at 85° C. using a mobile phase of 0.01 N sulfuric acid at a flow rate of 1.0 mL/min and a diode array detector at 315 nm for detection. Analytes were identified by comparing retention times and spectral profiles with pure standards.

### Example 6

### Calculation of Succinate Yields, Succinate Productivity, and Succinate Maximum Specific Productivity

Succinic acid (SA) yield is calculated as the ratio of SA (g) and total sugar consumption (g) at the end of the fermentation. Metabolic yield is calculated similarly to SA yield but correcting substrate and product concentrations with the dilution produced from base addition. Compensation was also made for the removal of substrate and products via sampling (considering 1 mL as sample volume). SA titer (g/L) is the SA concentration at the end of the fermentation and values are not corrected by the dilution factor. Overall productivity (g/L/h) is calculated as SA production (g/L) divided by the time (h) at the end of the fermentation. The end of the fermentation is considered when total sugar concentration is close to zero. Maximum instantaneous productivity (g/L/h) is the maximum productivity peak value observed during the fermentation course.

### Example 7

### Plasmid and Strain Construction

Q5 Hot Start High-Fidelity Polymerase and 2× Master Mix (New England Biolabs, MA) and primers were used in all PCR amplification for plasmid construction. Plasmids were constructed using restriction enzyme subcloning or Gibson Assembly Master Mix (New England Biolabs, MA) following the manufacturer's instructions. Plasmids were transformed into competent Zymo 5-alpha E. coli (Zymo Research, CA) following the manufacturer's instructions or electroporation into A. succinogenes. Transformants were selected on LB or TSB plates containing 10 g/L glucose, supplemented with either 50 μg/mL chloramphenicol or 50 μg/mL ampicillin and grown at 37° C. The sequences of all plasmid inserts were confirmed using Sanger sequencing.

### Example 8

Construction of Marker-Less Knockouts in A. succinogenes 130Z

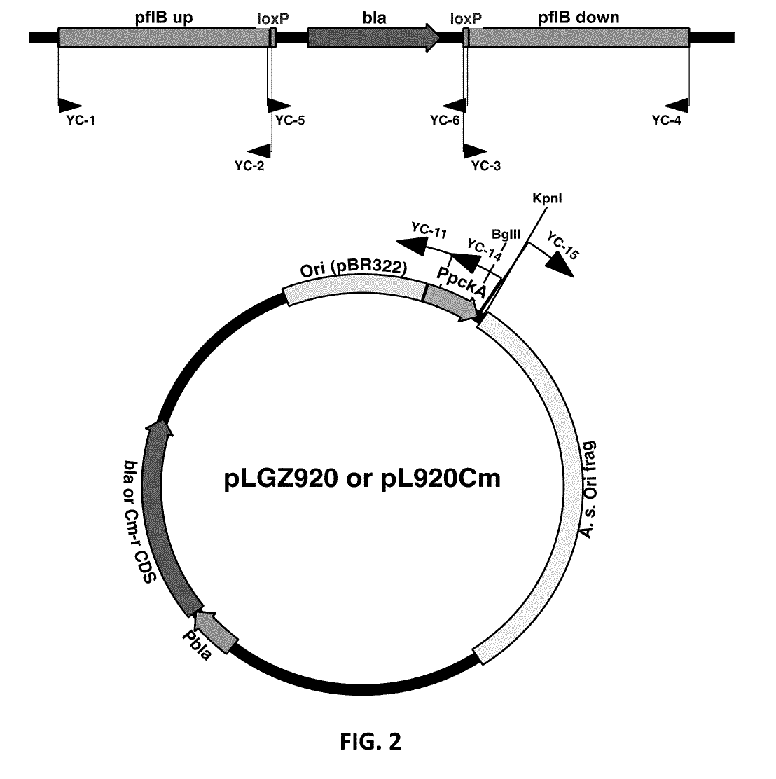

To knock out pflB and ackA in the genome of 130Z via homologous recombination, a knockout cassette was constructed (FIG. 2). DNA fragments including 1.4-kb and 1.5-kb up- and down-stream regions of target genes and an ampicillin resistance gene (bla) flanked by loxP sequences were individually amplified by polymerase chain reactions using primer sets indicated in Table 4. Three fragments were assembled using a Gibson Assembly Cloning kit (New England Biolabs, MA). The cassette was used to transform A. succinogenes host using electroporation and integrants were selected on TSBG plates supplemented with ampicillin. Integration was verified by PCR analysis and DNA sequencing. Ampicillin resistance gene in the pfl KO integrants was removed by transformation with a plasmid expressing Cre recombinase (pL920CreCm). A plasmid curing procedure was performed to further remove the pL920CreCm from the Amp-sensitive KO integrants, involving daily sub-culturing of integrants in liquid TSBG for over 25 generations followed by plating on TSBG agar and identifying the Amp-sensitive and Cm-sensitive colonies.

TABLE 4

| SEQ | | | |
|---|---|---|---|
| ID | Name | Primers (5′ to 3′) | Usage |
| 17 | YC-1 | CGTTAACCGTGGGAATCA | pflB up fragment; |
| | | GTTTGTTAGGAATG | Gibson Assembly |
| 18 | YC-2 | CGAAGTTATTGTAATACT | pflB up fragment; |
| | | TCCTTTTGCTAGTATTGA | Gibson Assembly |
| | | TAATGAAATCCTGTAAG | |
| 19 | YC-3 | CCATAACTTCGTATAATG | pflB down fragment; |
| | | TATGCTATACGAAGTTAT | Gibson Assembly |
| | | TTGGGGTAACGTAATAAA | |
| | | AATG | |
| 20 | YC-4 | TCTCTCCTTCGCGGAATA | pflB down fragment; |
| | | AAATATCCACTTC | Gibson Assembly |
| 21 | YC-5 | CTAGCAAAAGGAAGTATT | bla-loxP fragment; |
| | | ACAATAACTTCGTATAAT | Gibson Assembly |
| | | GTATGCTATACGAAGTTA | |
| | | TAATTCTTGAAGACGAAA | |
| | | GGGCCTCGTG | |
| 22 | YC-6 | CGTATAGCATACATTATA | bla-loxP fragment; |
| | | CGAAGTTATGGGGTCTGA | Gibson Assembly |
| | | CGCTCAGTGGAACGAAAA | |
| | | CTC | |
| 23 | YC-7 | GCAGCAATAGAGGAAACA | pckA fragment |
| | | CGGTTTG | |
| 24 | YC-8 | GGATTTGGTACCGTGCCG | pckA fragment |
| | | GCGGCCTAATAACCTG | |
| 25 | YC-9 | CGAACCGAAGCGTTCCTG | mdh fragment; |
| | | CGCGAGTAACGC | blunt-end ligation |
| 26 | YC-10 | GCTTCCCATTAATCAAAC | mdh fragment; |
| | | GGCGG | blunt-end ligation |
| 27 | YC-11 | CTCAAACAAACCGTGTTT | pLGZ920 to linearize |
| | | CCTCTATTGCTGC | for mdh cloning |
| 28 | YC-12 | AATTATCAATGAGGTGAA | fum CDS fragment; |
| | | GTATGACATTTCGTATTG | Gibson Assembly |
| | | AAAAAGACAC | |
| 29 | YC-13 | GAATTCGAGCTCGGTACC | fum CDS fragment; |
| | | CGGGGATCCCTGACCGTC | Gibson Assembly |
| | | TTCGGTGAATACTGATAT | |
| | | AG | |
| 30 | YC-14 | ACTTCACCTCATTGATAA | pLGZ920 to linearize |
| | | TTTAAAATTAAAAATCC | for fum cloning |
| 31 | YC-15 | GGATCCCCGGGTACCGAG | pLGZ920 to linearize |
| | | CTCGAATTCACTG | for mdh or fum |
| 32 | YC-16 | CGGTACCGAGCTCGAATT | pPCK to linearize |
| | | CACTGGCCGTCG | for mdh and fum |
| | | | cloning |
| 33 | YC-17 | GTCATCTTAACAGGTTAT | pPCK to linearize |
| | | TAGGCCGCCGGCA | for mdh and fum |
| | | | cloning |
| 34 | YC-18 | CCTTCGGCCGGCCCTGCC | fum fragment |
| | | GTTTCGGAAAACTCACGC | |
| | | TTTACCCG | |
| 35 | YC-19 | CCTTCGGCCGGCCCATAA | fum fragment |
| | | AGAATCCAAGATAAACGA | |
| | | ATTGGC | |

### Example 9

Construction of Gene Overexpression Strains of A. succinogenes 130Z

All plasmids were constructed based on an E. coli—A. succinogenes shuttle vector pLGZ920 (ATCC PTA-6140 and its derivative pL920Cm (FIG. 2). Overexpression plasmids are listed in Table 2. PCR using primers described in Table 4 was used to generate all DNA fragments. Restriction enzyme digestion or Gibson Assembly techniques were incorporated in the cloning of the genes. For single-gene overexpression constructs (pPCK, pMDH and pFUM), genes encoding PEP carboxykinase (pckA), malate dehydrogenase (mdh) and fumarase (fum) from A. succinogenes 130Z were independently cloned in pLGZ920. The promoter of pckA (PpckA) was used to drive the expression of pckA and fum whereas mdh was under the control of its own promoter. In the three-gene overexpression constructs, pPMF, pckA, mdh and fum were under the control of their respective native promoters.

### Example 10

Electroporation of A. succinogenes

Plasmids and/or linear DNA cassettes were transformed into A. succinogenes via electroporation using Gene Pulser Xcell (Bio-Rad, CA). Electro-competent cells were prepared by harvesting the exponential growth phase cells (OD₆₀₀=0.4-0.5) and centrifuging at 4° C., 3300×g for 10 minutes. Cells were washed twice in ½ volume of ice-cold 15% glycerol and concentrated 100× to 150× before electroporation. One hundred microliters of competent cells were mixed with 2-15 μL DNA in a 0.2-cm gap cuvette and electroporated at 2.5 kV, 25 μF and 600 ohms. One-milliliter TSBG was added to the cuvette after electroporation. Cells were transferred to a microtube and incubated at 37° C. for one hour before plating on TSBG agar plates supplemented with appropriate antibiotics. Plates were incubated at 37° C. for 1 to 2 days or until colonies were observed.

### Example 11

### Genetic Tool Development

While prior metabolic engineering efforts in A. succinogenes have employed a positive selection strategy that leveraged the organism's glutamate auxotrophy, an alternative set of positive selection tools were developed via the conventional utilization of antibiotic resistance markers. Electroporation of A. succinogenes with linear PCR fragments containing genomic homology regions flanking an antibiotic resistance marker enabled homologous recombination-mediated chromosomal integration and gene disruption (FIG. 2); flanking markers with loxP sites enables antibiotic marker removal and recycling via expression of Cre recombinase. Concurrent deployment in combination with a modified version of the pLGZ920 expression vector, encoding a chloramphenicol resistance gene (FIG. 2), enables facile gene overexpression in wild-type and knockout mutant backgrounds. Using these tools, a series of strains with altered carbon flux through central carbon metabolism and fermentation pathways were generated to examine alterations in SA biosynthesis (Table 2).

### Example 12

### Ablation of Competitive Carbon Pathway Components

Wild-type A. succinogenes splits carbon flux between two main fermentative pathways: a SA producing, reductive C4 pathway, and a number of oxidative C3 pathways that produce acetic acid, formic acid, and ethanol as byproducts. As SA is a highly reduced product, biosynthesis of these additional compounds serves in part to generate cellular energy and reducing power. However, from a process perspective, these byproducts also serve as competitive carbon sinks, potentially reducing carbon yields and/or flux to SA. Additionally, the presence of organic acids other than SA reduces separation efficiency and product purity in downstream recovery of SA from fermentation broth, serving as a negative techno-economic cost driver.

To examine the effects of removal of competitive carbon pathways in A. succinogenes, the targeted knockout of genetic components encoding biosynthetic machinery of C3 pathway products acetic and formic acid was conducted. Pyruvate formate lyase (pflB), which catalyzes the reversible conversion of pyruvate and coenzyme-A into formic acid and acetyl-CoA, and acetate kinase (ackA), which catalyzes conversion of acetyl-phosphate and ADP to acetate and ATP (FIG. 1), were chromosomally ablated to generate marker-less mutants (ΔpflB and ΔackA, respectively, Table 2). Additionally, a pflB, ackA double mutant was generated via iterative chromosomal integration and marker removal (strain ΔpflBΔackA).

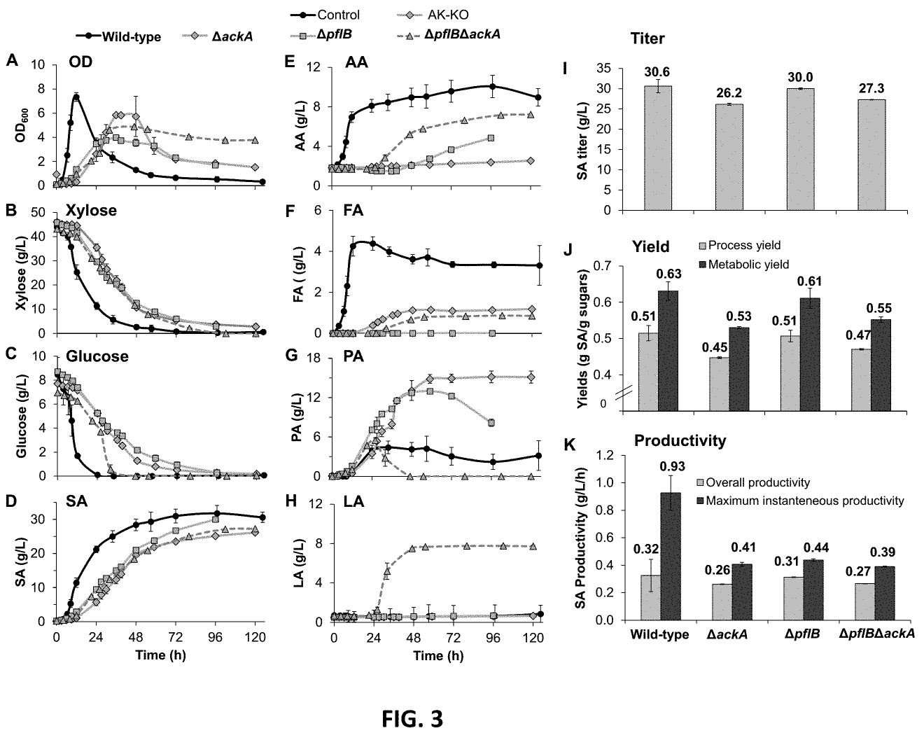

Wild-type and mutant A. succinogenes strains were cultivated on mock biomass hydrolysate containing a 60 g/L total sugars stream rich in xylose as a carbon source over a 96-hour batch fermentation. FIG. 3 shows different fermentation and metabolic parameters in wild-type A. succinogenes 130Z compared to the knockout strains ΔackA, ΔpflB, and ΔpflBΔackA. Scatter plots present (A) bacterial growth, utilization of (B) xylose and (C) glucose, and acid production: (D) SA, (E) acetic acid (AA), (F) formic acid (FA), (G) pyruvic acid (PA), and (H) lactic acid (LA). The bar graphs show (I) SA titers, (J) yields and metabolic yields, and (K) overall and maximum instantaneous productivities. SA “yield” is the ratio of SA (g/L) and the sugars consumed (g/L) at the end of the fermentation. SA “metabolic yield” is calculated as the yield considering the dilution factor at the end of the fermentation. The overall productivity was calculated at 96 hours in all cases (since sugars were mostly consumed at that time). The maximum instantaneous productivity was observed at 12 hours for the control and 48 hours for the knockout strains.

Both single and double mutants displayed a growth defect, demonstrating an additional 9-hour lag phase relative to wild-type (FIG. 3, panel A). Growth rates (μ⁻¹), calculated from the maximum slope before cell aggregation occurred, were 0.48, 0.15, 0.21, and 0.17 for the wild-type, ΔackA, ΔpflB, and ΔpflBΔackA, respectively. An associated delay in onset and rate of glucose and xylose consumption was also observed, though complete sugar consumption was ultimately achieved in all strains following 96 hours of cultivation (FIG. 3, panels B and C). For the other sugars present in the mock, about 0.4 g/L arabinose (from an initial 6.5 g/L) and 2.4 g/L galactose (from an initial 3.5 g/L), were remaining at the end of the fermentation in the four strains, demonstrating a degree of preferential carbon utilization. Delayed growth and sugar consumption also coincided with delayed onset of SA biosynthesis relative to wild-type (FIG. 3, panel D). SA titer, yield, and overall productivity (FIG. 3, panels I, J, and K) decreased in ΔackA and ΔpflBΔackA compared to wild-type and ΔpflB strains. The latter may have a reduced effect on metabolism due to the partially redundant action of pyruvate dehydrogenase in carrying flux to acetyl-CoA.

Although overall productivity was similar between ΔpflB and wild-type strains, the maximum instantaneous productivity was lower in ΔpflB (FIG. 3, panel K) due to the initial lag in bacterial growth and thus, organic acid production. As shown in FIG. 3, panel G, strains lacking acetate kinase activity compensated for the loss in reducing power by accumulating pyruvate, which resulted in a net production of 2 mol of NADH per mol of glucose. As this is lower than the 4 mol of NADH produced per mol of glucose with acetate and CO₂ as the final products (FIG. 1), more carbon was drawn to the oxidative metabolic branches, resulting in a lower SA yield. These results indicate the removal of heterofermentative pathways is insufficient to enhance carbon flux to SA under the cultivation conditions examined here.

Despite delayed growth and onset of SA biosynthesis, effective reduction in acetate and formate was observed in all three mutants (FIG. 3, panels E and F), and nearly complete elimination of acetate and formate was observed in ΔackA and ΔpflB single mutants, respectively. Reduction of acetate was observed in ΔpflB mutants, and conversely, reduction in formate was observed in ΔackA mutants, indicative of interrelated and/or interdependent energetic and redox regulation of these biosynthetic pathways. Further, despite removal of the canonical acetate kinase-mediated biosynthetic pathway, acetic acid accumulation was still observed in the ΔpflBΔackA strain, indicating that an alternative route to acetic acid biosynthesis exists in A. succinogenes. Acetyl-CoA synthetase (acs)-mediated acetic acid biosynthesis, which offers a direct route from acetyl-CoA to acetic acid, does not appear to explain these findings.

Additional pyruvate accumulation was observed in both ΔackA and ΔpflB single mutants, indicating a flux bottleneck upon removal of the acetate and formate carbon sinks, respectively (FIG. 3, panel G). Conversely, the ΔpflBΔackA strain showed pyruvate accumulation profiles similar to wild-type, yet ultimately displayed complete pyruvate reassimilation capacity following 24 hours of cultivation. Notably, this pyruvate reassimilation coincided with lactate production (FIG. 3, panel H); no lactic acid accumulation was observed in wild-type or single mutant strains, indicating the double mutant channels more reductant to lactic acid compared to the wild-type and engineered strains examined. As lactic acid production involves the oxidation of NADH, this additional product likely explains the reduced SA yield observed in double knockout strains. Ethanol was not detected in any fermentation, even though its production also serves to regenerate NAD⁺.

### Example 13

### Overexpression of Succinic Acid Biosynthetic Machinery

As an alternative means to enhance flux to SA, overexpression of SA biosynthetic machinery was examined. Wild-type A. succinogenes channels carbon through the reductive branch of the TCA cycle (FIG. 1), proceeding through oxaloacetate, malate, and fumarate intermediates via the activity of phosphoenolpyruvate carboxykinase (PCK), malate dehydrogenase (MDH), fumarase (FUM), and fumarate reductase, respectively. PCK in part facilitates the capnophilic activity of A. succinogenes via carboxylation of phosphoenolpyruvate to oxaloacetate. A series of strains overexpressing reductive TCA pathway genes were generated to examine the relative flux impact of these biosynthetic components.

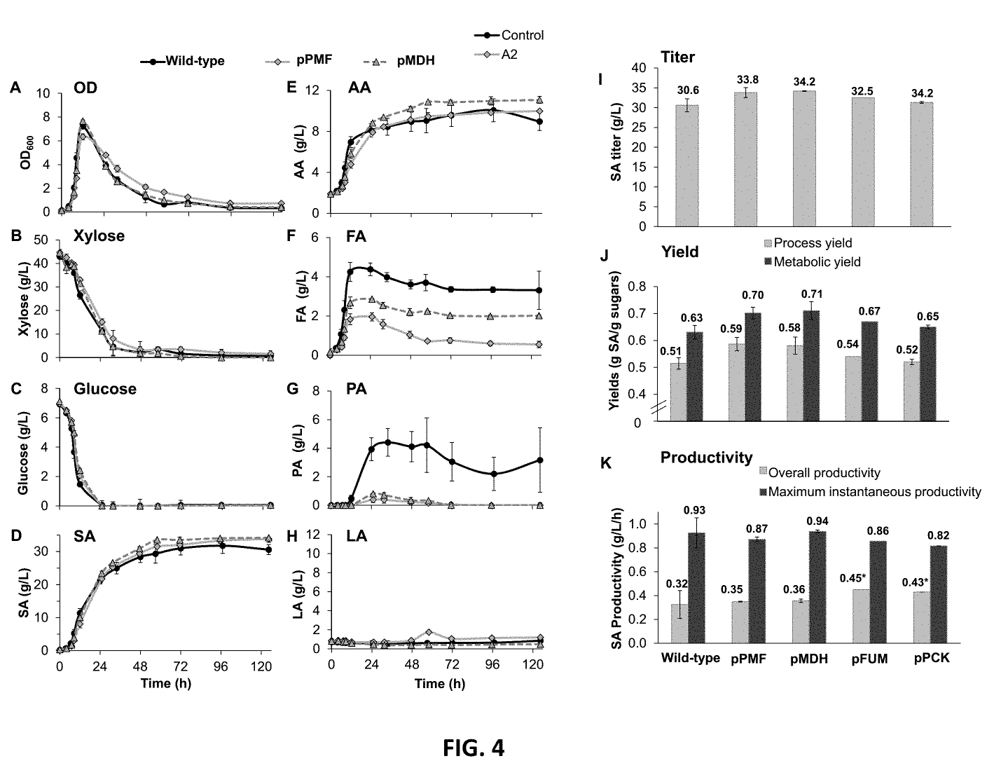

FIG. 4 shows different fermentation and metabolic parameters in wild-type A. succinogenes (130Z) compared to overexpression constructs pMDH and pPFM. The scatter plots show (A) bacterial growth, utilization of (B) xylose and (C) glucose, and acid production: (D) SA, (E) acetic acid (AA), (F) formic acid (FA), (G) pyruvic acid (PA), and (H) lactic acid (LA). The bar graphs show (I) SA titers, (J) yields and metabolic yields, and (K) overall and maximum instantaneous productivities. SA “yield” is calculated as the ratio of SA (g/L) and the sugars consumed (g/L) at the end of the fermentation. SA “metabolic yield” is calculated as the yield but considering the dilution factor at the end of the fermentation. The overall productivity was calculated at 96 hours in all cases (since sugars were mostly consumed at that time). The maximum instantaneous productivity was observed at 12 hours for the control and 25 hours for the overexpression strains.

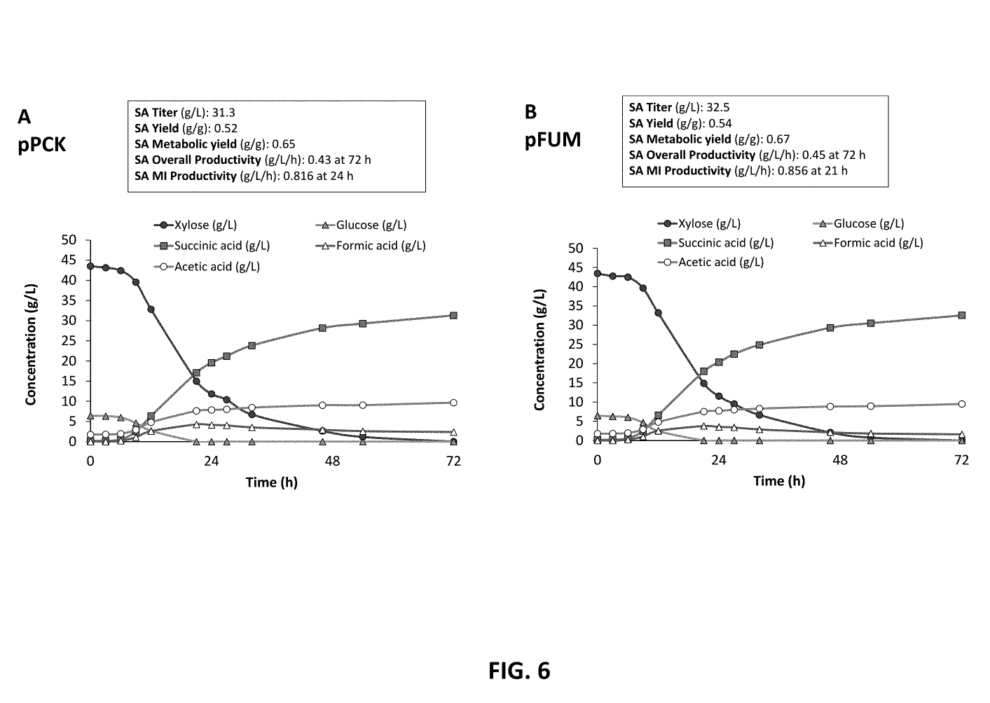

Initially, three single gene overexpression strains were compared. The three strains, overexpressing PCK (pPCK, FIG. 6), MDH (pMDH, FIG. 4), and FUM (pFUM, FIG. 6) increased titers (31.3, 34.2, 32.6 g/L, respectively) and metabolic yields (0.65, 0.71, and 0.67 g/g, respectively) compared to the wild-type (30.6 g/L and 0.51 g/g, respectively). We also generated a strain overexpressing three components of the reductive TCA branch, PCK, MDH, and FUM (pPMF). Considering the SA titer enhancement conferred by MDH gene overexpression, the pMDH strain was further compared with wild-type and pPMF (FIG. 4). Growth rates and sugar utilization profiles for all strains were similar (FIG. 4, panels A and B). While initial onset of SA biosynthesis was similar for all strains (FIG. 4, panel C), the overexpression strains displayed superior SA accumulation capacity relative to wild-type, with the pMDH overexpression strain demonstrating the highest titer of 34.2 g/L, an 11.8% titer enhancement (FIG. 4, panel I).

In addition to enhanced SA titers, all three overexpression strains also exhibit higher final titers of acetic acid relative to wild-type (FIG. 4, panel E), indicating acetic acid biosynthesis is a component in achieving higher SA concentrations. Strains with enhanced SA flux may balance the additional need for reducing power with the production of acetate, as the production of acetic acid and CO₂ results in the highest yields of NADH per mol of glucose (FIG. 1). The formic acid concentration was significantly higher in the wild-type strain compared to the overexpression strains during the cultivation (FIG. 4, panel F). In addition, a greater reduction in formic acid titer is observed in the pPMF overexpression strain relative to wild-type thereafter, with levels similar to those found in both ΔackA and ΔpflBΔackA strains (FIG. 4, panel F; FIG. 3, panel F). This indicates the strain preferentially utilizes PFL over pyruvate dehydrogenase, and further oxidizes formate to CO₂ later in the fermentation. Minimal pyruvate accumulation (less than 1 g/L) was observed in overexpression strains (FIG. 4, panel G). Conversely, wild-type A. succinogenes accumulated nearly six-fold higher pyruvate at 58 hours of cultivation. Lactic acid production was only observed in the pPMF strain at low levels and a single time point (FIG. 4, panel H). Ethanol was not produced by any of the strains.

Titer and yield enhancements were observed for all overexpression strains (FIG. 4, panels I and J). Overall and maximum instantaneous productivity values were similar for the wild-type and engineered strains. In addition to the titer enhancement, 12.70% and 9.1% maximum metabolic yield and overall productivity increases were observed, respectively, in the top-performing strain, pMDH (FIG. 4, panels I, J, and K). Flux redirection therefore appears more effective in A. succinogenes by pulling carbon towards reduction pathways via gene overexpression, as opposed to forcing carbon into reduction pathways via marker-less gene knockout of the oxidative branch of metabolism. Expression of MDH alone generated the maximum SA titer, yield, and production rate (FIG. 4), indicating necessity and sufficiency for biosynthetic enhancement, and implicating oxaloacetate to malate conversion as the rate-limiting step in the reductive TCA cycle of A. succinogenes. Carbon balance analyses further validate these findings (FIG. 8), demonstrating nearly complete carbon closure of top performing strains, with equivalent product-to-biomass distribution ratios, and nearly complete metabolite identification.

### Example 14

### Concurrent Succinic Acid Biosynthesis Gene Overexpression and Competitive Carbon Pathway Ablation

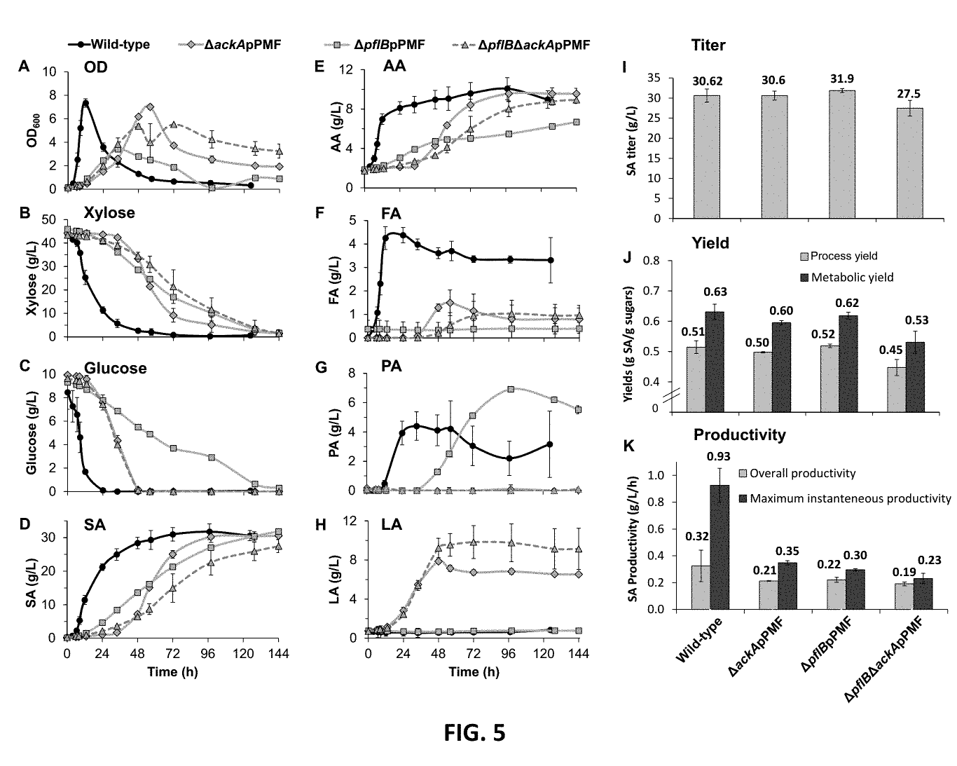

The effects of incorporating pPMF overexpression into three mutant backgrounds (ΔpflB, ΔackA, and ΔpflBΔackA) was investigated. pPMF was selected for further combinatorial engineering to ensure maximum flux through the reductive branch of the pathway in combination with knockouts in the oxidative branch. FIG. 5 shows different fermentation and metabolic parameters in wild-type A. succinogenes (130Z) compared to ΔackA/pPMF, ΔpflB/pPMF, and ΔpflBΔackA/pPMF strains. The scatter plots show (A) bacterial growth, utilization of (B) xylose and (C) glucose, and acid production: (D) SA, (E) acetic acid (AA), (F) formic acid (FA), (G) pyruvic acid (PA), and (H) lactic acid (LA). The bar graphs show (I) SA titers, (J) yields and metabolic yields, and (K) overall and maximum instantaneous productivities. SA “yield” is calculated as the ratio of SA (g/L) and the sugars consumed (g/L) at the end of the fermentation. SA “metabolic yield” is calculated as the yield but considering the dilution factor at the end of the fermentation. The overall productivity was calculated at 96 hours in the wild-type and at 144 hours in the rest of the strains. The maximum instantaneous productivity was observed at 12 hours for the control, about 72 hours for ΔackA/pPMF, ΔpflB/pPMF, and 98 hours for ΔpflBΔackA/pPMF.

As with the above described knockout mutant strains (FIG. 3), a growth defect and concurrent delay in SA onset was observed (FIG. 5, panel A). The growth rates (μ⁻¹) were 0.48, 0.10, 0.14, and 0.13 for the wild-type control, ΔackA/pPMF, ΔpflB/pPMF, and ΔpflBΔackA/pPMF strains, respectively. A uniform lag in xylose consumption was observed for all engineered strains relative to wild-type (FIG. 5, panel B). Glucose consumption was also delayed in all three strains, with pPMF overexpression in the ΔpflB background displaying the highest glucose consumption defect (FIG. 5, panel C). Despite this decreased rate of glucose consumption, the ΔpflB/pPMF strain displayed the highest final SA titer among the combinatorially engineered strains (FIG. 5, panel D). Regarding maximum SA production rates, the wild-type produced SA at 1.75 g/L/h between 8.5-12 hours, followed by ΔackA/pPMF, which produced SA at rates of 0.98 g/L/h between 48-56 hours (FIG. 5, panel D). ΔpflB/pPMF and ΔpflBΔackA/pPMF produced SA at maximum rates of 0.38 g/L/h between 24-34 hours and 56-72 hours, respectively (FIG. 5, panel D). Combined, these data indicate xylose consumption is a sufficient driver for SA biosynthesis and further support a role for acetate co-production in SA biosynthesis in A. succinogenes.

The acetic acid production rate lagged in the mutant backgrounds, as observed for mutants in the absence of MDH overexpression. Acetic acid accumulation was again observed in all ackA mutant backgrounds, further supporting an alternative route to acetic acid in A. succinogenes (FIG. 5, panel E). As in mutant and overexpression strains described above, formate reduction was also observed in all combinatorial strains (FIG. 5, panel F). Similarly, an initial reduction in pyruvate was observed in combinatorially engineered strains relative to wild-type, although the ΔpflB/pPMF strain displayed significant pyruvate accumulation following 24 hours of cultivation. The wild-type strain displays pyruvate reassimilation capacity, whereas the ΔpflB/pPMF strain does not (FIG. 5, panel G). Lactic acid is generated in both the acetate kinase and double-mutant backgrounds with pPMF overexpression (FIG. 5, panel H). This differs from the mutant backgrounds with native SA biosynthetic machinery intact (no overexpression of reductive TCA components), demonstrating a fine-tuned interplay between organic acid biosynthesis and TCA cycle flux. Ablation of both ackA and pflB appears necessary for lactate production in the absence of additional flux alterations, whereas ackA knockout is sufficient for lactate production when concurrent TCA component overexpression is employed.

Final SA titers were enhanced (4%) in the ΔpflB/pPMF strain relative to wild-type, with minimal impact on yield (FIG. 5, panels I and J), again underscoring the role of overexpression of reductive TCA components in enhancement of SA accumulation. However, productivity was decreased in all combinatorially engineered strains. Overall productivity was calculated at the time that all the sugars were totally consumed, corresponding to 96 and 144 hours for the wild-type and combinatorially engineered strains, respectively. Due to this fact, although SA titers were similar at 144 hours (FIG. 5, panel I), the productivity was lower for the engineered strains (FIG. 5, panel K). A similar effect is observed in the maximum instantaneous productivity. The wild-type production peaked at 12 hours, while single mutant backgrounds peaked at 72 hours, and the double mutant background at 98 hours.

### Example 15

### Additional Engineered Strains

Strains of A. succinogenes were also engineered to overexpress either phosphoenolpyruvate carboxykinase (pPCK) or fumarase (pFUM). FIG. 6 shows different fermentation and metabolic parameters in (A) pPCK and (B) pFUM strains. Scatter plots present utilization of xylose and glucose, and acids production such as SA, acetic acid, and formic acid. In the boxes, SA titers, yields and metabolic yields, and overall and maximum instantaneous productivities are presented. SA “yield” is calculated as the coefficient of SA (g/L) and the sugars consumed (g/L) at the end of the fermention. SA “metabolic yield” is calculated as the yield but considering the dilution factor at the end of the fermentation.

Strains of A. succinogenes were also engineered to overexpress phosphoglucose dehydrogenase in wild type strains (pGDH) or in strains where pyruvate formate lyase has also been knocked out (ΔpflBpGDH). FIG. 7 shows different fermentation and metabolic parameters in (A) pGDH and (B) ΔpflBpGDH strains. Scatter plots present bacterial growth, utilization of xylose and glucose, and acids production such as SA, acetic acid, formic acid, and pyruvic acid. In the boxes, growth rates, SA titers, yields and metabolic yields, and overall and maximum instantaneous productivities are presented. SA “yield” is calculated as the coefficient of SA (g/L) and the sugars consumed (g/L) at the end of the fermention. SA “metabolic yield” is calculated as the yield but considering the dilution factor at the end of the fermentation.

The Examples discussed above are provided for purposes of illustration and are not intended to be limiting. Still other embodiments and modifications are also contemplated.

While a number of exemplary aspects and embodiments have been discussed above, those of skill in the art will recognize certain modifications, permutations, additions and sub combinations thereof. It is therefore intended that the following appended claims and claims hereafter introduced are interpreted to include all such modifications, permutations, additions and sub-combinations as are within their true spirit and scope.

We claim:

1. An engineered cell, comprising at least one exogenously added gene encoding an enzyme from the reductive branch of the tricarboxylic acid (TCA) cycle, wherein the cell is able to produce succinic acid from a carbon source.

2. The engineered cell of claim 1, wherein the exogenously added gene encodes malate dehydrogenase, PEP-carboxykinase, or fumarase.

3. The engineered cell of claim 1, wherein the exogenously added gene encodes malate dehydrogenase.

4. The engineered cell of claim 3, further comprising an additional exogenously added gene encoding PEP-carboxykinase, fumarase, or both.

5. The engineered cell of claim 1, wherein the cell is a bacterial cell.

6. The engineered cell of claim 5, wherein the bacterial cell is from the genus Actinobacillus.

7. The engineered cell of claim 6, wherein the bacterial cell is from Actinobacillus succinogenes.

8. The engineered cell of claim 7, wherein the bacterial cell is from Actinobacillus succinogenes strain 130Z.

9. The engineered cell of claim 1, wherein the cell produces a higher amount of succinic acid than the wild type cell.

10. The engineered cell of claim 1, wherein the carbon source is derived from lignocellulosic biomass.

11. The engineered cell of claim 10, wherein the carbon source comprises glucose, xylose, galactose or arabinose.

12. The engineered cell of claim 1, further comprising a genetic modification that reduces the production of acetate or formate by the cell.

13. The engineered cell of claim 12, wherein the genetic modification reduces the expression of pyruvate formate lyase or acetate kinase.

14. The engineered cell of claim 12, wherein the genetic modification reduces the expression of pyruvate formate lyase.

15. The engineered cell of claim 1, further comprising an exogenously added gene encoding XylE or phosphoglucose dehydrogenase.

16. A method for producing succinic acid, comprising:
a) culturing the engineered cell of claim 1 with a carbon source; and
b) recovering the succinic acid from the culture.

17. The method of claim 16, wherein the carbon source is derived from lignocellulosic biomass.

18. The method of claim 16, wherein the engineered cell comprises an exogenously added gene encoding malate dehydrogenase.

19. The method of claim 18, wherein the engineered cell further comprises a genetic modification that reduces the expression of pyruvate formate lyase or acetate kinase.

20. The method of claim 16, wherein the engineered cell is a bacterial cell from Actinobacillus succinogenes.
